(19) United States
(12) Patent Application Publication
(10) Pub. No.: US 2018/0036302 A1
(43) Pub. Date: Feb. 8, 2018

(54) TREATMENT OF MULTIPLE SCLEROSIS WITH COMBINATION OF LAQUINIMOD AND A STATIN

(71) Applicants: Victor Piryatinsky, Netanya (IL); Joel Kaye, Netanya (IL)

(72) Inventors: Victor Piryatinsky, Netanya (IL); Joel Kaye, Netanya (IL)

(73) Assignee: Teva Pharmaceutical Industries Ltd., Petach Tikva (IL)

(21) Appl. No.: 15/533,612

(22) PCT Filed: Dec. 9, 2015
(86) PCT No.: PCT/US2015/064705

§ 371 Date: Jun. 6, 2017

Related U.S. Application Data
(60) Provisional application No. 62/090,112, filed on Dec. 10, 2014.

(51) Int. Cl. A61K 31/4704; A61K 9/28; A61K 47/26; A61K 45/06; A61K 47/12; A61K 47/02; A61K 9/14; A61K 31/40; A61K 47/18

(52) U.S. Cl. CPC A61K 31/4704; A61K 31/40; A61K 9/2866; A61K 47/26; A61K 47/18; A61K 47/12; A61K 47/02; A61K 9/14; A61K 45/06

(57) ABSTRACT

This invention provides a method of treating a subject afflicted with multiple sclerosis (MS) or presenting a clinically isolated syndrome (CIS) comprising administering to the subject laquinimod as an add-on to or in combination with a statin. This invention also provides a package and a pharmaceutical composition comprising laquinimod and a statin for treating a subject afflicted with MS or presenting CIS. This invention also provides laquinimod for use as an add-on therapy or in combination with a statin in treating a subject afflicted with MS or presenting CIS. This invention further provides use of laquinimod and a statin in the preparation of a combination for treating a subject afflicted with MS or presenting CIS.

This application claims priority of U.S. Provisional Application No. 62/090,112, filed Dec. 10, 2014, the entire content of which is hereby incorporated by reference herein.

Throughout this application, various publications are referred to by first author and year of publication. Full citations for these publications are presented in a References section immediately before the claims. The disclosures of these documents and publications referred to herein are hereby incorporated in their entireties by reference into this application in order to more fully describe the state of the art to which this invention pertains.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesions go on to develop MS, while approximately 20 percent have a self-limited process (Brex 2002; Frohman, 2003).

Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and Fingolimod (Gilenya®). Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

### Statins

Statins are a class of drugs that are widely prescribed in the management and prevention of cardiovascular disease. Studies have suggested that statins can lower low-density lipoprotein (LDL) cholesterol levels by up to 55% and cardiovascular events by 20-30% (Postmus, 2014).

Statins are 3-hydroxy-3-methylglutaryl-coenzyme A (HMG CoA) reductase inhibitors. HMG CoA reductase is the rate-limiting enzyme in cholesterol synthesis. By competitively inhibiting HMG CoA reductase activity, statins decrease cellular cholesterol concentration, which activates a cellular signaling cascade culminating in the activation of sterol regulatory element binding protein (SREBP). SREBP is a transcription factor that up-regulates expression of the gene encoding the LDL receptor. LDL receptors are responsible for receptor-mediated endocytosis of LDL cholesterol. Thus, increased LDL receptor expression causes increased uptake of plasma LDL and consequently decrease plasma LDL-cholesterol concentration (Armen, 2012).

The best-selling statin drug is atorvastatin, marketed as LIPITOR® and manufactured by Pfizer. Lipitor® is available in tablet form for daily oral administration, each tablet containing 10, 20, 40, or 80 mg atorvastatin (Physician's Desk Reference, 2014).

In addition to Lipitor, statins are also commercially available as single-ingredient products as Lescol® (fluvastatin), Mevacor® (lovastatin), Altoprev® (lovastatin extended-release), Livalo® (pitavastatin), Pravachol® (pravastatin), Crestor® (rosuvastatin), and Zocor® (simvastatin). Statins are also commercially available as combination products as Advicor® (lovastatin/niacin extended-release), Simcor® (simvastatin/niacin extended-release), and Vytorin® (simvastatin/ezetimibe) (Statins, 2012).

### Laquinimod

Laquinimod (TV-5600) is a novel synthetic compound with high oral bioavailability which has been suggested as an oral formulation for the treatment of Multiple Sclerosis (MS) (Polman, 2005; Sandberg-Wollheim, 2005; Comi at al 2008). Laquinimod and its sodium salt form are described, for example, in U.S. Pat. No. 6,077,851. The mechanism of action of laquinimod is not fully understood.

Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2006; Brück, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

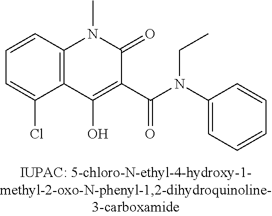 [SMILES: CCN(C(=O)c1c(O)c2c(Cl)cccc2n(C)c1=O)c1ccccc1]

### Combination Therapy

The administration of two drugs to treat a given condition, such as multiple sclerosis, raises a number of potential problems. In vivo interactions between two drugs are complex. The effects of any single drug are related to its absorption, distribution, metabolism, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 2012). In one example, combined administration of fingolimod and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 2012). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 2012).

Therefore, the state of the art at the time of filing is that the effects of combination therapy of two drugs, in particular laquinimod and a statin, e.g., atorvastatin, cannot be predicted until the results of a combination study are available.

### BRIEF DESCRIPTION OF THE DRAWINGS

FIG. 1 is a graphical representation of the experimental results from Example 1. The graph shows the clinical score for the EAE rodents in each group (on the y-axis) against the days after induction of the disease (on the x-axis).

FIG. 2 is a graphical representation of the experimental results from Example 2: Mean plasma concentration-time profiles of laquinimod after oral dose of laquinimod alone or combination dose with atorvastatin in male C57BL/6 mice (N=3/time point).

FIG. 3 is a graphical representation of the experimental results from Example 2: Mean plasma concentration-time profiles of atorvastatin after oral dose of atorvastatin alone or combination dose with laquinimod in male C57BL/6 mice (N=3/time point).

### SUMMARY OF THE INVENTION

The subject invention provides a method of treating a subject afflicted with multiple sclerosis (MS) or presenting a clinically isolated syndrome (CIS) comprising administering to the subject an amount of laquinimod and administering to the subject an amount of a statin.

The subject invention also provides a package comprising: a) a first pharmaceutical composition comprising an amount of laquinimod and a pharmaceutically acceptable carrier; b) a second pharmaceutical composition comprising an amount of a statin and a pharmaceutically acceptable carrier; and c) instructions for use of the first and second pharmaceutical compositions together to treat a subject afflicted with MS or presenting a CIS.

The subject invention also provides a therapeutic package for dispensing to, or for use in dispensing to, a subject afflicted with MS or presenting a CIS, which comprises: a) one or more unit doses, each such unit dose comprising: i) an amount of laquinimod and ii) an amount of a statin, wherein the respective amounts of said laquinimod and said statin in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

The subject invention also provides a pharmaceutical composition comprising an amount of laquinimod and an amount of a statin.

The subject invention also provides a process of preparing a pharmaceutical composition comprising an amount of laquinimod and an amount of a statin, comprising 1) obtaining an amount of laquinimod and an amount of a statin, and 2) admixing the laquinimod and the statin with a pharmaceutically acceptable carrier to make the pharmaceutical composition.

The subject invention also provides a pharmaceutical composition in unit dosage form, useful in treating a subject afflicted with MS or presenting a CIS, which comprises: a) an amount of laquinimod; b) an amount of a statin, wherein the respective amounts of said laquinimod and said statin in said composition are effective, upon concomitant administration to said subject of one or more of said unit dosage forms of said composition, to treat the subject.

The subject invention also provides a pharmaceutical composition comprising an amount of laquinimod for use in treating a subject afflicted with MS or presenting a CIS as an add-on therapy or in combination with a statin.

The subject invention also provides a pharmaceutical composition comprising an amount of laquinimod for use in treating a subject afflicted with MS or presenting a CIS simultaneously, contemporaneously or concomitantly with a statin.

The subject invention also provides a process of preparing a pharmaceutical composition prepared for treating a subject afflicted with MS or presenting a CIS using an amount of laquinimod, either as an add-on therapy to or in combination with an amount of a statin, comprising 1) obtaining an amount of laquinimod, and 2) admixing the laquinimod with a pharmaceutically acceptable carrier.

The subject invention also provides a pharmaceutical composition comprising an amount of a statin for use treating a subject afflicted with MS or presenting a CIS as an add-on therapy or in combination with laquinimod.

The subject invention also provides a pharmaceutical composition comprising an amount of a statin for use treating a subject afflicted with MS or presenting a CIS simultaneously, contemporaneously or concomitantly with laquinimod.

The subject invention also provides laquinimod for use as an add-on therapy or in combination with a statin in treating a subject afflicted with MS or presenting a CIS.

The subject invention also provides a statin for use as an add-on therapy or in combination with laquinimod in treating a subject afflicted with MS or presenting a CIS.

The subject invention also provides use of an amount of laquinimod and an amount of a statin in the preparation of a combination for treating a subject afflicted with MS or presenting a CIS wherein the laquinimod and the statin are prepared to be administered simultaneously, contemporaneously or concomitantly.

The subject invention also provides use of an amount of laquinimod in the manufacture of a medicament for treating a subject afflicted with MS or presenting a CIS wherein the laquinimod is prepared as an add-on therapy to or in combination with an amount of a statin, and wherein the amount of laquinimod and the amount of statin when taken together are effective to treat the subject.

The subject invention also provides use of an amount of laquinimod and an amount of a statin in the manufacture of a medicament for treating a subject afflicted with MS or presenting a CIS, wherein the amount of laquinimod and an amount of statin when taken together are effective to treat the subject.

The subject invention also provides a process of preparing a medicament prepared for treating a subject afflicted with MS or presenting a CIS using an amount of laquinimod, either as an add-on therapy to or in combination with an amount of a statin, comprising 1) obtaining a pharmaceutical composition comprising an amount of laquinimod and a pharmaceutically acceptable carrier, and 2) packaging the pharmaceutical composition to make the medicament.

The subject invention also provides a process of preparing a medicament prepared for treating a subject afflicted with MS or presenting a CIS using an amount of laquinimod and an amount of a statin, comprising 1) obtaining a pharmaceutical composition comprising an amount of laquinimod, an amount of a statin, and a pharmaceutically acceptable carrier, and 2) packaging the pharmaceutical composition to make the medicament.

### DETAILED DESCRIPTION OF THE INVENTION

The subject invention provides a method of treating a subject afflicted with multiple sclerosis (MS) or presenting a clinically isolated syndrome (CIS) comprising administering to the subject an amount of laquinimod and administering to the subject an amount of a statin.

In one embodiment, the amount of laquinimod and the amount of the statin when taken together is more effective to treat the subject than when each agent at the same respective amount is administered alone.

In an embodiment, the MS is relapsing MS. In another embodiment, the relapsing MS is relapsing-remitting MS.

In one embodiment, the amount of laquinimod and the amount of the statin when taken together is effective to reduce a symptom of MS in the subject. In another embodiment, the symptom is a MRI-monitored MS disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

In one embodiment, the amount of laquinimod and the amount of the statin when taken together is effective to a) decrease or inhibit reduction of brain volume, b) increase time to confirmed disease progression, c) decrease abnormalities observed in whole Brain MTR histogram, or d) reduce cognitive impairment.

In an embodiment, brain volume is measured by percent brain volume change (PBVC). In another embodiment, time to confirmed disease progression is increased by 20-60%. In another embodiment, cognitive impairment is assessed by the Symbol Digit Modalities Test (SDMT) score. In another embodiment, the accumulation of physical disability is measured by Kurtzke Expanded Disability Status Scale (EDSS) score, or is assessed by the time to confirmed disease progression as measured by EDSS score.

In one embodiment, the subject had an EDSS score of 0-5.5 at baseline, an EDSS score of 1.5-4.5 at baseline or an EDSS score of 5.5 or greater at baseline.

In another embodiment, confirmed disease progression is a 1 point or a 0.5 point increase of the EDSS score.

In one embodiment, impaired mobility is assessed by the Timed-25 Foot Walk test, the 12-Item MS Walking Scale (MSWS-12) self-report questionnaire, the Ambulation Index (AI), the Six-Minute Walk (6MW) Test or the Lower Extremity Manual Muscle Test (LEMMT) Test. In another embodiment, general health status is assessed by the EuroQoL (EQ5D) questionnaire, Subject Global Impression (SGI) or Clinician Global Impression of Change (CGIC). In another embodiment, functional status is measured by the subject's Short-Form General Health survey (SF-36) Subject Reported Questionnaire score. In another embodiment, quality of life is assessed by SF-36, EQ5D, Subject Global Impression (SGI) or Clinician Global Impression of Change (CGIC). In another embodiment, the subject's SF-36 mental component summary score (MSC) is improved. In another embodiment, the subject's SF-36 physical component summary sore (PSC) is improved. In another embodiment, fatigue is assessed by the EQ5D, the subject's Modified Fatigue Impact Scale (MFIS) score or the French valid versions of the Fatigue Impact Scale (EMIF-SEP) score. In another embodiment, symptom severity on work is measured by the work productivity and activities impairment General Health (WPAI-GH) questionnaire.

In a further embodiment of the present invention, laquinimod is laquinimod sodium. In another embodiment, the statin is atorvastatin calcium.

In one embodiment of the present invention, the laquinimod and/or the statin is administered via oral administration. In another embodiment, the laquinimod and/or the statin is administered periodically. In another embodiment, the laquinimod and/or the statin is administered daily. In another embodiment, the laquinimod and/or the statin is administered more often than once daily. In another embodiment, the laquinimod and/or the statin is administered less often than once daily. In another embodiment, the amount of laquinimod administered is less than 0.6 mg/day. In another embodiment, the amount of laquinimod administered is 0.1-40.0 mg/day. In another embodiment, the amount of laquinimod administered is 0.1-2.5 mg/day. In another embodiment, the amount of laquinimod administered is 0.25-2.0 mg/day. In another embodiment, the amount of laquinimod administered is 0.5-1.2 mg/day. In another embodiment, the amount of laquinimod administered is 0.25 mg/day, 0.3 mg/day, 0.5 mg/day, 0.6 mg/day, 1.0 mg/day, 1.2 mg/day, 1.5 mg/day or 2.0 mg/day.

In one embodiment, the amount of the statin administered is 0.1-100 mg/day. In another embodiment, the amount of the statin administered is 10-80 mg/day. In another embodiment, the amount of statin administered is about 10, 20, 40, or 80 mg/day. In another embodiment, the amount of the statin administered is 10, 20, 40, or 80 mg/day.

In an embodiment, a loading dose of an amount different from the intended dose is administered for a period of time at the start of the periodic administration. In another embodiment, the subject is receiving laquinimod therapy prior to initiating the statin therapy. In another embodiment, the subject is receiving the statin therapy prior to initiating laquinimod therapy. In another embodiment, the subject is receiving a first therapy for at least 8 weeks, at least 10 weeks, at least 24 weeks, at least 28 weeks, at least 48 weeks or at least 52 weeks prior to initiating a second therapy.

In a further embodiment of the present invention, the method further comprises administration of nonsteroidal anti-inflammatory drugs (NSAIDs), salicylates, slow-acting drugs, gold compounds, hydroxychloroquine, sulfasalazine, corticosteroids, cytotoxic drugs, immunosuppressive drugs and/or antibodies.

In an embodiment, the periodic administration of laquinimod and/or the periodic administration of the statin continues for at least 3 days, for more than 30 days, for more than 42 days, for 8 weeks or more, for at least 12 weeks, for at least 24 weeks or for 6 months or more.

In an embodiment, the administration of laquinimod and the administration of the statin inhibits a symptom of relapsing MS by at least 20%, by at least 30%, by at least 50%, by at least 70%, by more than 100%, by more than 300% or by more than 1000%.

In an embodiment, each of the amount of laquinimod when taken alone, and the amount of the statin when taken alone is effective to treat the subject. In another embodiment, either the amount of laquinimod when taken alone, the amount of the statin when taken alone, or each such amount when taken alone is not effective to treat the subject.

In an embodiment, the subject is a human patient.

The subject invention also provides a package comprising: a) a first pharmaceutical composition comprising an amount of laquinimod and a pharmaceutically acceptable carrier; b) a second pharmaceutical composition comprising an amount of a statin and a pharmaceutically acceptable carrier; and c) instructions for use of the first and second pharmaceutical compositions together to treat a subject afflicted with MS or presenting a CIS.

In one embodiment, the first pharmaceutical composition, the second pharmaceutical composition, or both the first and the second pharmaceutical compositions are in an aerosol, an inhalable powder, an injectable, a liquid, a solid, a capsule or a tablet form. In another embodiment, the first pharmaceutical composition, the second pharmaceutical composition, or both the first and the second pharmaceutical compositions are in a liquid or a solid form. In yet another embodiment, the first pharmaceutical composition, the second pharmaceutical composition, or both the first and the second pharmaceutical compositions are in capsule form or in tablet form.

In one embodiment, the tablets are coated with a coating which inhibits oxygen from contacting the core. In another embodiment, the coating comprises a cellulosic polymer, a detackifier, a gloss enhancer, or pigment.

In an embodiment, the first pharmaceutical composition further comprises mannitol, an alkalinizing agent, an oxidation reducing agent, a lubricant, and/or a filler. In another embodiment, the alkalinizing agent is meglumine. In another embodiment, the lubricant is present in the composition as solid particles. In another embodiment, the lubricant is sodium stearyl fumarate or magnesium stearate. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In yet another embodiment, the filler is mannitol or lactose monohydrate.

In one embodiment, the first pharmaceutical composition is stable and free of an alkalinizing agent or an oxidation reducing agent. In another embodiment, the first pharmaceutical composition is free of an alkalinizing agent and free of an oxidation reducing agent. In a further embodiment, the first pharmaceutical composition is stable and free of disintegrant.

In one embodiment, the package further comprises a desiccant. In another embodiment, the desiccant is silica gel.

In a further embodiment of the present invention, the first pharmaceutical composition is stable and has a moisture content of no more than 4%.

In one embodiment, laquinimod is present in the composition as solid particles.

In another embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle and/or comprises an HDPE bottle. In another embodiment, the bottle is closed with a heat induction liner. In another embodiment, the sealed package comprises an oxygen absorbing agent. In yet another embodiment, the oxygen absorbing agent is iron.

In one embodiment, the amount of laquinimod in the first composition is less than 0.6 mg. In another embodiment, the amount of laquinimod in the first composition is 0.1-40.0 mg. In another embodiment the amount of laquinimod is 0.1-2.5 mg. In another embodiment the amount of laquinimod is 0.25-2.0 mg. In another embodiment the amount of laquinimod is 0.5-1.2 mg. In another embodiment the amount of laquinimod is 0.25 mg, 0.3 mg, 0.5 mg, 0.6 mg, 1.0 mg, 1.2 mg, 1.5 mg or 2.0 mg.

In one embodiment, the amount of the statin is 0.1-100 mg. In another embodiment, the amount of the statin is 10-80 mg. In another embodiment, the amount of the statin is about 10, 20, 40 or 80 mg. In another embodiment, the amount of the statin is 10, 20, 40 or 80 mg.

In an embodiment, the amount of laquinimod and the amount of the statin are prepared to be administered simultaneously, contemporaneously or concomitantly.

The subject invention also provides a therapeutic package for dispensing to, or for use in dispensing to, a subject afflicted with MS or presenting a CIS, which comprises: a) one or more unit doses, each such unit dose comprising: i) an amount of laquinimod and ii) an amount of a statin, wherein the respective amounts of said laquinimod and said statin in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

In one embodiment, the respective amounts of said laquinimod and said statin in said unit dose when taken together is more effective to treat the subject than when compared to the administration of said laquinimod in the absence of the statin or the administration of the statin in the absence of said laquinimod. In another embodiment, the statin is atorvastatin calcium.

The subject invention also provides a pharmaceutical composition comprising an amount of laquinimod and an amount of a statin. In an embodiment, the pharmaceutical composition consists essentially of an amount of laquinimod and an amount of a statin.

In an embodiment, the pharmaceutical composition is for use in treating a subject afflicted with MS or presenting a CIS, wherein the laquinimod and the statin are prepared to be administered simultaneously, contemporaneously or concomitantly.

In one embodiment, laquinimod is laquinimod sodium. In another embodiment, the statin is atorvastatin calcium.

In a further embodiment, the pharmaceutical composition is in an aerosol, an inhalable powder, an injectable, a liquid, a solid, a capsule or a tablet form. In another embodiment, the tablets are coated with a coating which inhibits oxygen from contacting the core. In another embodiment, the coating comprises a cellulosic polymer, a detackifier, a gloss enhancer, or pigment.

In a further embodiment, the pharmaceutical composition further comprises mannitol, an alkalinizing agent, an oxidation reducing agent, a lubricant or a filler. In another embodiment, the alkalinizing agent is meglumine. In another embodiment, the lubricant is present in the composition as solid particles. In another embodiment, the lubricant is sodium stearyl fumarate or magnesium stearate. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In yet another embodiment, the filler is mannitol or lactose monohydrate.

In one embodiment, the pharmaceutical composition is free of an alkalinizing agent or an oxidation reducing agent. In another embodiment, it is free of an alkalinizing agent and free of an oxidation reducing agent. In yet another embodiment, it is stable and free of disintegrant. In another embodiment, the amount of laquinimod in the composition is less than 0.6 mg. In another embodiment, the amount of laquinimod in the composition is 0.1-40.0 mg. In another embodiment, the amount of laquinimod is 0.1-2.5 mg. In another embodiment, the amount of laquinimod is 0.25-2.0 mg. In another embodiment, the amount of laquinimod is 0.5-1.2 mg. In another embodiment, the amount of laquinimod is 0.25 mg, 0.3 mg, 0.5 mg, 0.6 mg, 1.0 mg, 1.2 mg, 1.5 mg, 2.0 mg.

In an embodiment, the amount of the statin is 0.1-100 mg. In another embodiment, the amount of the statin is 10-80 mg. In another embodiment, the amount of the statin is about 10, 20, 40 or 80 mg. In another embodiment, the amount of the statin is 10, 20, 40 or 80 mg.

The subject invention also provides a process of preparing a pharmaceutical composition comprising an amount of laquinimod and an amount of a statin, comprising 1) obtaining an amount of laquinimod and an amount of a statin, and 2) admixing the laquinimod and the statin with a pharmaceutically acceptable carrier to make the pharmaceutical composition.

The subject invention also provides a pharmaceutical composition in unit dosage form, useful in treating a subject afflicted with MS or presenting a CIS, which comprises: a) an amount of laquinimod; b) an amount of a statin, wherein the respective amounts of said laquinimod and said statin in said composition are effective, upon concomitant administration to said subject of one or more of said unit dosage forms of said composition, to treat the subject. In an embodiment, the respective amounts of said laquinimod and the statin in said unit dose when taken together is more effective to treat the subject than when compared to the administration of said laquinimod in the absence of the statin or the administration of the statin in the absence of said laquinimod.

The subject invention also provides a pharmaceutical composition comprising an amount of laquinimod for use in treating a subject afflicted with MS or presenting a CIS as an add-on therapy or in combination with a statin.

The subject invention also provides a pharmaceutical composition comprising an amount of laquinimod for use in treating a subject afflicted with MS or presenting a CIS simultaneously, contemporaneously or concomitantly with a statin.

The subject invention also provides a process of preparing a pharmaceutical composition prepared for treating a subject afflicted with MS or presenting a CIS using an amount of laquinimod, either as an add-on therapy to or in combination with an amount of a statin, comprising 1) obtaining an amount of laquinimod, and 2) admixing the laquinimod with a pharmaceutically acceptable carrier.

The subject invention also provides a pharmaceutical composition comprising an amount of a statin for use treating a subject afflicted with MS or presenting a CIS as an add-on therapy or in combination with laquinimod.

The subject invention also provides a pharmaceutical composition comprising an amount of a statin for use treating a subject afflicted with MS or presenting a CIS simultaneously, contemporaneously or concomitantly with laquinimod.

The subject invention also provides laquinimod for use as an add-on therapy or in combination with a statin in treating a subject afflicted with MS or presenting a CIS.

The subject invention also provides a statin for use as an add-on therapy or in combination with laquinimod in treating a subject afflicted with MS or presenting a CIS.

The subject invention also provides use of an amount of laquinimod and an amount of a statin in the preparation of a combination for treating a subject afflicted with MS or presenting a CIS wherein the laquinimod and the statin are prepared to be administered simultaneously, contemporaneously or concomitantly.

In an embodiment of the pharmaceutical composition or use as described herein, the statin is atorvastatin.

The subject invention also provides use of an amount of laquinimod in the manufacture of a medicament for treating a subject afflicted with MS or presenting a CIS wherein the laquinimod is prepared as an add-on therapy to or in combination with an amount of a statin, and wherein the amount of laquinimod and the amount of statin when taken together are effective to treat the subject.

The subject invention also provides use of an amount of laquinimod and an amount of a statin in the manufacture of a medicament for treating a subject afflicted with MS or presenting a CIS, wherein the amount of laquinimod and an amount of statin when taken together are effective to treat the subject.

The subject invention also provides a process of preparing a medicament prepared for treating a subject afflicted with MS or presenting a CIS using an amount of laquinimod, either as an add-on therapy to or in combination with an amount of a statin, comprising 1) obtaining a pharmaceutical composition comprising an amount of laquinimod and a pharmaceutically acceptable carrier, and 2) packaging the pharmaceutical composition to make the medicament.

The subject invention also provides a process of preparing a medicament prepared for treating a subject afflicted with MS or presenting a CIS using an amount of laquinimod and an amount of a statin, comprising 1) obtaining a pharmaceutical composition comprising an amount of laquinimod, an amount of a statin, and a pharmaceutically acceptable carrier, and 2) packaging the pharmaceutical composition to make the medicament.

The statins as described herein can be administered by way of oral, sublingual, injection including subcutaneous, intramuscular and intravenous, topical, intratracheal, intranasal, transdermal or rectal administration. The statins may be administered in admixture with conventional pharmaceutical carriers. The appropriate unit forms of administration include forms for oral administration, such as tablets, gelatin capsules, powders, granules and solutions or suspensions to be taken orally, forms for sublingual, buccal, intratracheal or intranasal administration, forms for injection including subcutaneous, intramuscular or intravenous administration and forms for rectal administration. In one particular embodiment, oral administration is preferred.

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in its entireties into this application.

Use of laquinimod for treatment of various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzheimer's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in its entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit can be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the unit dosage form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and WO 2007/146248.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds); Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject, e.g., human patient, afflicted with multiple sclerosis, e.g., relapsing multiple sclerosis or presenting a CIS using laquinimod with a statin such as atorvastatin which provides a more efficacious treatment than each agent alone. The use of laquinimod for multiple sclerosis had been previously suggested in, e.g., U.S. Pat. No. 6,077,851. However, the inventors have surprisingly found that the combination of laquinimod and statin such as atorvastatin is particularly effective for the treatment of a subject afflicted with MS or presenting a CIS as compared to each agent alone.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

### Terms

As used herein, and unless stated otherwise, each of the following terms shall have the definition set forth below.

As used herein, “laquinimod” means laquinimod acid or a pharmaceutically acceptable salt thereof.

As used herein, a “statin”, “3-hydroxy-3-methylglutaryl-coenzyme A reductase inhibitor” or “HMG CoA reductase inhibitor” is an agent which inhibits the activity of 3-hydroxy-3-methylglutaryl-coenzyme A reductase enzyme. Examples of statins include atorvastatin (Lipitor®) lovastatin (Mevacor®, Altoprev®), pravastatin (Pravochol®), fluvastatin (Lescol®), simvastatin (Zocor®), Rosuvastatin (Crestor®), pitavastatin (Livalo®), mevastatin, cerivastatin, velostatin, fluindostatin, dalvastatin, rivastatin, eptastatin, itavastatin, nisvastatin, compctin and dihydrocompactin. In addition as used herein the term “statin” includes a pharmaceutically acceptable salt thereof.

A “salt” is salt of the instant compounds which have been modified by making acid or base salts of the compounds. The term “pharmaceutically acceptable salt” in this respect, refers to the relatively non-toxic, inorganic and organic acid or base addition salts of compounds of the present invention. Examples of salts of the instant compounds include sodium salts and calcium salts of said compounds.

As used herein, an “amount” or “dose” of laquinimod or statin as measured in milligrams refers to the milligrams of laquinimod acid or statin present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, a “unit dose”, “unit doses” and “unit dosage form(s)” mean a single drug administration entity/entities.

As used herein, “about” in the context of a numerical value or range means ±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound or molecule that inhibits the oxidation of other molecules. Examples of antoxidants include tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein is also exemplified by flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound exemplified by the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound exemplified by the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the laquinimod and the statin. In this case, the combination may be the admixture or separate containers of the laquinimod and the statin that are combined just prior to administration. Contemporaneous administration refers to the separate administration of the laquinimod and the statin at the same time, or at times sufficiently close together that a additive or preferably synergistic activity relative to the activity of either the laquinimod or the statin alone is observed.

As used herein, “concomitant administration” or administering “concomitantly” means the administration of two agents given in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving atorvastatin therapy.

As used herein, “effective” when referring to an amount of laquinimod and/or statin refers to the quantity of laquinimod and/or statin that is sufficient to yield a desired therapeutic response. Efficacy can be measured by an improvement of a symptom of multiple sclerosis. Such symptoms can include a MRI-monitored multiple sclerosis disease activity, relapse rate, accumulation of physical disability, frequency of relapses, time to confirmed disease progression, time to confirmed relapse, frequency of clinical exacerbation, brain atrophy, neuronal dysfunction, neuronal injury, neuronal degeneration, neuronal apoptosis, risk for confirmed progression, visual function, fatigue, impaired mobility, cognitive impairment, brain volume, abnormalities observed in whole Brain MTR histogram, general health status, functional status, quality of life, and/or symptom severity on work.

In an embodiment, an effective amount is an amount that is sufficient to decrease or inhibit reduction of brain volume (optionally brain volume is measured by percent brain volume change (PBVC)), increase time to confirmed disease progression (e.g., by 20-60% or at least 50%), decrease abnormalities observed in whole Brain MTR histogram, decrease the accumulation of physical disability (optionally measured by Kurtzke Expanded Disability Status Scale (EDSS) score, e.g., wherein the accumulation of physical disability is assessed by the time to confirmed disease progression as measured by Kurtzke Expanded Disability Status Scale (EDSS) score), improve impaired mobility (optionally assessed by the Timed-25 Foot Walk test, the 12-Item Multiple Sclerosis Walking Scale (MSWS-12) self-report questionnaire, the Ambulation Index (AI), the Six-Minute Walk (6MW) Test, or the Lower Extremity Manual Muscle Test (LEMMT) Test), reduce cognitive impairment (optionally assessed by the Symbol Digit Modalities Test (SDHT) score), improve general health (optionally assessed by the EuroQoL (EQ5D) questionnaire, Subject Global Impression (SGI) or Clinician Global Impression of Change (CGIC)), improve functional status (optionally measured by the subject's Short-Form General Health survey (SF-36) Subject Reported Questionnaire score), improve quality of life (optionally assessed by SF-36, EQ5D, Subject Global Impression (SGI) or Clinician Global Impression of Change (CGIC)), improve the subject's SF-36 mental component summary score (MSC) and/or SF-36 physical component summary sore (PSC), reduce level of fatigue (optionally assessed by the EQ5D, the subject's Modified Fatigue Impact Scale (MFIS) score or the French valid versions of the Fatigue Impact Scale (EMIF-SEP) score), or improve symptom severity on work (optionally measured by the work productivity and activities impairment General Health (WPAI-GH) questionnaire).

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times a week and so on, etc.

“Treating” as used herein encompasses, e.g., inducing inhibition, regression, or stasis of a disease or disorder, e.g., Relapsing MS (RMS), or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with MS or RMS includes any clinical or laboratory manifestation associated with MS or RMS and is not limited to what the subject can feel or observe.

As used herein, “a subject afflicted with multiple sclerosis” or “a subject afflicted with relapsing multiple sclerosis” means a subject who has been clinically diagnosed to have multiple sclerosis or relapsing multiple sclerosis (RMS), which includes relapsing-remitting multiple sclerosis (RRMS) and Secondary Progressive multiple sclerosis (SPMS).

As used herein, a subject at “baseline” is as subject prior to administration of laquinimod and the statin as described herein.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4⁺ T cells, CD8⁺ T cells, anti-NF-L, anti-CSF 114(Glc)).

As used herein “multiple sclerosis” include each of the five distinct disease stages and/or types of MS: 1) benign multiple sclerosis; 2) RRMS 3) SPMS; 4) progressive relapsing multiple sclerosis (PRMS); and 5) primary progressive multiple sclerosis (PPHS).
1) Benign multiple sclerosis is a retrospective diagnosis characterized by 1-2 exacerbations with complete recovery, no lasting disability and no disease progression for 10-15 years after the initial onset. Benign multiple sclerosis may, however, progress into other forms of multiple sclerosis.
2) Patients suffering from RRMS experience sporadic exacerbations or relapses, as well as periods of remission. Lesions and evidence of axonal loss may or may not be visible on MRI for patients with RRMS.
3) SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS.
4) PPMS is characterized by a steady progression of increasing neurological deficits without distinct attacks or remissions. Cerebral lesions, diffuse spinal cord damage and evidence of axonal loss are evident on the MRI of patients with PPMS. PPMS has periods of acute exacerbations while proceeding along a course of increasing neurological deficits without remissions. Lesions are evident on MRI of patients suffering from PRMS. (Johnson et al., 1986).

Multiple sclerosis may present with optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning.

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter. Patients who experience a single clinical attack consistent with MS may have at least one lesion consistent with MS prior to the development of clinically definite MS.

The term relapsing MS includes 1) patients with RRMS; 2) patients with SPMS and superimposed relapses; and 3) patients with CIS who show lesion dissemination on subsequent MRI scans according to McDonald's criteria. As used herein, the term “relapsing MS” or “relapsing forms of multiple sclerosis” include: 1) RRMS, characterized by unpredictable acute episodes of neurological dysfunction (relapses), followed by variable recovery and periods of clinical stability; 2) SPMS, wherein patients having RRMS develop sustained deterioration with or without relapses superimposed; and 3) PPRMS or PRMS, an uncommon form wherein patients developing a progressive deterioration from the beginning can also develop relapses later on.

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system for quantifying disability in MS, and is frequently used for classifying and standardizing the condition of people with multiple sclerosis. The EDSS replaced the previous Disability Status Scales which used to bunch people with MS in the lower brackets. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS) (Kurtzke J F, 1983).

A “confirmed progression” of EDSS, or “confirmed disease progression” as measured by EDSS score is defined as a 1 point increase from baseline EDSS if baseline EDSS was between 0 and 5.0, or a 0.5 point increase if baseline EDSS was 5.5. In order to be considered a confirmed progression, the change (either 1 point or 0.5 points) must be sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

“Gd-enhancing lesion” refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

“Magnetization Transfer Imaging” or “MTI” is based on the magnetization interaction (through dipolar and/or chemical exchange) between bulk water protons and macromolecular protons. By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom. With MTI, the presence or absence of macromolecules (e.g. in membranes or brain tissue) can be seen (Mehta, 1996; Grossman, 1994).

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism (Golder, 2007).

As used herein “mobility” refers to any ability relating to walking, walking speed, gait, strength of leg muscles, leg function and the ability to move with or without assistance. Mobility can be evaluated by one or more of several tests including but not limited to Ambulation Index, Time 25 foot walk, Six-Minute Walk (6MW), Lower Extremity Manual Muscle Test (LEMMT) and EDSS. Mobility can also be reported by the subject, for example by questionnaires, including but not limited to 12-Item Multiple Sclerosis Walking Scale (MSWS-12). Impaired Mobility refers to any impairment, difficulty or disability relating to mobility.

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

The “Six-Minute Walk (6MW) Test” is a commonly used test developed to assess exercise capacity in patients with COPD (Guyatt, 1985). It has been used also to measure mobility in multiple sclerosis patients (Clinical Trials Website).

The “Timed-25 Foot Walk” or “T25-FW” is a quantitative mobility and leg function performance test based on a timed 25-walk. The patient is directed to one end of a clearly marked 25-foot course and is instructed to walk 25 feet as quickly as possible, but safely. The time is calculated from the initiation of the instruction to start and ends when the patient has reached the 25-foot mark. The task is immediately administered again by having the patient walk back the same distance. Patients may use assistive devices when doing this task. The score for the T25-FW is the average of the two completed trials. This score can be used individually or used as part of the MSFC composite score (National MS Society Website).

One of the central symptoms of multiple sclerosis is fatigue. Fatigue can be measured by several tests including but not limited to decrease of French valid versions of the Fatigue Impact Scale (EMIF-SEP) score, and European Quality of Life (EuroQoL) Questionnaire (EQ5D). Other tests, including but not limited to Clinician Global Impression of Change (CGIC) and Subject Global Impression (SGI), as well as EQ-5D, can be used to evaluate the general health status and quality of life of MS patients.

“Ambulation Index” or “AI” is a rating scale developed by Hauser et al. to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. (Hauser, 1983)

“EQ-5D” is a standardized questionnaire instrument for use as a measure of health outcome applicable to a range of health conditions and treatments. It provides a simple descriptive profile and a single index value for health status that can be used in the clinical and economic evaluation of health care as well as population health surveys. EQ-5D was developed by the “EuroQoL” Group which comprises a network of international, multilingual, multidisciplinary researchers, originally from seven centers in England, Finland, the Netherlands, Norway and Sweden. The EQ-5D questionnaire is in the public domain and can be obtained from EuroQoL.

“SF-36” is a multi-purpose, short-form health survey with 36 questions which yields an 8-scale profile of functional health and well-being scores as well as psychometrically-based physical and mental health summary measures and a preference-based health utility index. It is a generic measure, as opposed to one that targets a specific age, disease, or treatment group. The survey is developed by and can be obtained from QualityMetric, Inc. of Providence, R.I.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1: The Efficacy of Combination of Laquinimod and Atorvastatin IN MOG Induced EAE in C57BL Mice

### 1. Study Rationale and Objectives

The objective of this study was to test the suppressive activity of laquinimod in combination with Atorvastatin in the MOG induced chronic Experimental Autoimmune Encephalomyelitis (EAE) model in C57Bl/6 mice. The C57Bl/6 strain of mouse was selected, as it is an established chronic EAE model to test for the efficacy of candidate molecules for the treatment of Multiple sclerosis (MS).

### 2. Materials and Methods

### 2.1 Test Articles and Reagents

Atorvastatin
Vehicle for Atorvastatin (0.5% Methocel)
Laquinimod
Pertusis toxin, “Sigma”, Code #2980
Myelin Oligodendrocyte Lipoprotein Novatide (MOG-35-55)
Complete Freund's Adjuvant (CFA) “Sigma”, code: F-5881
Mycobacterium tuberculosis H37RA (MT) Mnf: Difco, code: 231141
Sterile phosphate buffered saline
Sterile purified water

### 2.2 Test System

Healthy, nulliparous, non-pregnant female mice of the C57Bl/6 strain were obtained. The animals weighed about 17-20 g on arrival, and were approximately 7 weeks of age. The body weights of the animals was recorded on the day of delivery. Overtly healthy animals were assigned to study groups arbitrarily before treatment commenced.

### 3. Experimental Procedure

### 3.1 EAE Induction

Active EAE was induced on day 1 by the subcutaneous injection in the flanks at 2 injection sites, the encephalitogenic mixture (emulsion) consisting of MOG and commercial CFA containing 5 mg/mL Mycobacterium tuberculosis (MT) at a volume of 0.2 mL/mouse in the right flank of the animals.

The dose of the MOG and MT is 300 μg/mouse and 500 μg/mouse respectively.

Pertussis toxin was injected intra peritoneally on the day of induction and 48 hours later at dose level of 150 ng/0.2 ml/mouse.

### 3.2 Experimental Design

The mice were allocated to the following treatment groups (15 mice/group):

TABLE 1
Treatment Groups

| | Treatment | | | Administration | Admin. |
|---|---|---|---|---|---|
| Group | groups | Dose/day | | Route | Period |
| 1 | Vehicle | 0.2 | ml/mouse | Gavage bid(AM/PM) | From Day 1-30 |
| | (0.5% MC) | | | | |
| 2 | 0.5% MC | 0.2 | ml/mouse | Gavage qd (AM) | From Day 1-30 |
| | Laquinimod | 5 | mg/kg/day | Gavage qd (PM) | |
| 3 | 0.5% MC | 0.2 | ml/mouse | Gavage qd (AM) | From Day 1-30 |
| | Laquinimod | 25 | mg/kg/day | Gavage qd (PM) | |
| 4 | Atorvastatin | 10 | mg/kg/day | Gavage qd(AM) | From Day 1-30 |
| | 0.5% MC | 0.2 | ml/mouse | Gavage qd (PM) | |
| 5 | Atorvastatin | 50 | mg/kg/day | Gavage qd(AM) | From Day 1-30 |
| | 0.5% MC | 0.2 | ml/mouse | Gavage qd (PM) | |
| 6 | Atorvastatin | 10 | mg/kg/day | Gavage qd(AM) | From Day 1-30 |
| | Laquinimod | 5 | mg/kg/day | Gavage qd (PM) | |
| 7 | Atorvastatin | 50 | mg/kg/day | Gavage qd(AM) | From Day 1-30 |
| | LAQUINIMOD | 5 | mg/kg/day | Gavage qd (PM) | |

AM-Morning;
PM-Evening

### 3.3 Preparation and Administration of Encephalitogenic Emulsion

Oil Portion: CFA (containing 5 mg/ml NT)
Liquid portion: 70 mg MOG was dissolved in 23.33 ml Normal saline to yield 3 mg/ml MOG.
Emulsification: The emulsion was made from equal parts of oil (23.33 mL CFA containing 5.0 mg/ml MT) and liquid portions (70 mg HOG/23.33 mL PBS) in 2 syringes connected to each other with Leur lock. The concentration of HOG in emulsion was 1.5 mg/mL. The emulsion was transferred to insulin syringe before injection. A 0.2 ml emulsion was injected into the flanks of each mouse in the study at 2 injection sites.

### 3.4 Preparation and Administration of Pertussis Toxin

98 μL Pertussis toxin (200 μg/ml) was added to 26 ml PBS to yield 750 ng/ml.

The pertussis toxin was administered intravenous on the day of encephalitogen injection and 48 hours later (150.0 ng/0.2 ml/mouse×2-300 ng/mouse).

### 3.5 Test Formulations (Preparation and Administration)

3.5.1 Laquinimod Concentration of 0.5 and 2.5 mg/ml laquinimod were prepared in 0.5% Methocel. The test formulations were stored at 2-8° C. until use in amber colored bottles for not more than 8 days.

The mice were administered an oral dose (gavage) from day 1, once daily (qd) with concentrations of laquinimod of 0.5 or 2.5 mg/ml a volume dose level of 200 μl/mouse by the oral route for a dose levels of 5 (groups #2, 6 and 7) or 25 mg/kg (group #3) according to experimental design in Table 1.

### 3.5.2 Atorvastatin

Formulations of Atorvastatin were prepared daily in 0.5% Methocel/H₂O.

Concentrations of 1 mg/ml (groups #4 and 6) and 5 mg/ml (groups #5 and 7) were prepared for dose levels of 10 and 50 mg/kg qd according to Table 1.

### 4. Experimental Observations

4.1 Morbidity and Mortality All animals were examined once daily to detect if any were dead or moribund.

### 4.2 EAE Clinical Signs

Scoring of EAE clinical signs was initiated on the 10th day post-EAE induction and continued daily for 30 days.

The clinical signs were recorded on observation cards according to a grading system described in Table 2.

TABLE 2
Evaluation of the EAE Clinical Signs

| Score | Signs | Description |
|---|---|---|
| 0 | Normal behavior | No neurological signs. |
| 1 | Limp tail | The distal part or the whole tail is limp and droops. |
| 2 | Righting reflex | Animal has difficulties to return on his feet when |
| | decrease | it is laid on his back. |
| 3 | Ataxia | wobbly walk - when the mouse walks the hind legs are |
| | | unsteady. |
| 4 | Early paralysis | The mouse has difficulties standing on its hind legs |
| | | but still has remnants of movement. |
| 5 | Full paralysis | The mouse can't move its legs at all, it looks |
| | | thinner and emaciated. |
| 6 | Moribund/Death | |

All mice having scores of 1 and above are considered sick. Animals with score 5 for more than 3 days are given score 6 and sacrificed for humane reasons. For calculation purposes, the score (6) of animals that are sacrificed or died is carried forward.

### 5. Data Analysis and Calculations

### 5.1 Acceptance Criteria for EAE Induced Negative Control Group

The control group should have at least 70% incidence. The MMS should be more than 2.0.

### 5.2 Calculation of the Incidence of Disease (Disease Ratio)

The number of sick animals in each group are summed.

The incidence of disease is calculated as:

$$INCIDENCE of DISEASE = ( \frac{No . of sick mice in treated group}{No . of sick mice in control group} )$$

The percent inhibition according to incidence is calculated as:

$$INHIBITION ( \% ) of INCIDENCE = ( 1 - \frac{Number of sick mice in treated group}{Number of sick mice in control group} ) \times 100$$

### 5.3 Calculation of the Mortality/Moribundity Rate (Mortality Ratio)

The number of dead or moribund animals in each group are summed.

The mortality of disease is calculated as:

$$MORTALITY of DISEASE = ( \frac{No . of dead or moribound mice in treated group}{No . of dead or moribound mice in control group} )$$

The percent inhibition according to mortality is calculated as:

$$INHIBITION ( \% ) of MORTALITY = ( 1 - \frac{Number of dead or moribound mice in treated group}{Number of dead or moribound mice in control group} ) \times 100$$

### 5.4 Calculation of Duration of Disease

The mean duration of disease expressed in days is calculated as:

$$Mean Duration = ( \frac{Σ Duration of disease of each mouse}{No . of mice in the group} )$$

### 5.5 Calculation of Mean Delay in Onset of Disease

The mean onset of disease expressed in days is calculated as:

$$Mean Onset = ( \frac{Σ Onset of disease of each mouse}{No . of mice in the group} )$$

The onset of disease for a mouse that did not develop EAE is considered as 31 days (one day after termination of study).

The mean delay in onset of disease expressed in days is calculated by subtracting the mean onset of disease in control group from test group.

### 5.6 Calculation of the Mean Maximal Score and Percent Inhibition

The mean maximal score (1MS) of each group is calculated as:

$$M M S = ( \frac{Σ Maximal Score of each mouse}{No . of mice in the group} )$$

The percent inhibition according to MMS is calculated as:

$$INHIBITION ( \% ) of M M S = ( 1 - \frac{M M S of treated group}{M M S of control group} ) \times 100$$

### 5.7 Calculation of the Group Mean Score and Percent Inhibition

The daily scores of each mouse in the test group are summed and the individual mean daily score (IMS) is calculated as:

$$I M S = ( \frac{Σ Daily score of mouse}{Observation period ( days )} )$$

The mean group score (GMS) is calculated as:

$$G M S = ( \frac{Σ I M S of each mouse}{No . of mice in the group} )$$

The percent inhibition is calculated as:

$$INHIBITION ( \% ) of G M S = ( 1 - \frac{G M S of treated group}{G M S of control group} ) \times 100$$

### 6. Results

### 6.1 Summary Table

A summary of the incidence, mortality, MMS, GMS, duration of the disease, onset of the disease, and the activity of each group compared to the vehicle treated control group is shown in Table 3.

### 6.2 The Clinical Profile

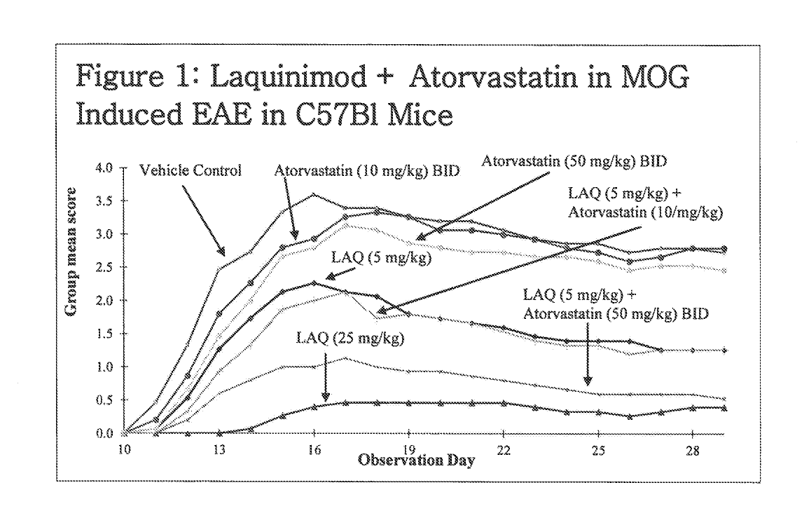

The clinical profile of the treatment groups are presented graphically in FIG. 1.

### 6.3 Additive Effect

Atorvastatin at dose level of 50 mg/kg when combined with laquinimod at dose level of 5 mg/kg exhibited additive effect expressed by greater activity according to Incidence, MMS, GMS, onset and Duration of EAE in group treated with combination of laquinimod (5 mg/kg) and Atorvastatin (50 mg/kg) compared to each tested alone.

TABLE 3
Summary Test Results
Test Articles: Laquinimod and Atorvastatin:
Mortality, Incidence, MMS, GMS, Duration, Onset, and EAE Inhibition Compared to Vehicle

| | | | % | | % | | % | | |
|---|---|---|---|---|---|---|---|---|---|
| | | | inhibition | MMS | inhibition | GMS | inhibition | Onset | Duration |
| Treatment | Moratality | Incidence | 1 | value | 2 | value | 3 | (days) | (days) |
| Vehicle | 0/15 | 15/15 | NA | 3.7 ± 0.5 | NA | 2.7 ± 0.4 | NA | 11.7 ± 0.7 | 19.3 ± 0.7 |
| (0.5% MC) | | | | | | | | | |
| LAQUINIMOD | 0/15 | 12/15 | 20% | 2.3 ± 1.3 | 37.8% | 1.4 ± 1.0 | 48.1% | 16.3 ± 7.7 | 13.5 ± 7.9 |
| 5 mg/kg | | | | | | | p < 0.01 | | |
| LAQUINIMOD | 0/15 | 5/15 | 66.7% | 0.7 ± 1.0 | 81.1% | 0.3 ± 0.6 | 91.9% | 25.63 ± 7.5 | 4.1 ± 6.0 |
| 25 mg/kg | | | | | | | p < 0.001 | | |
| Atorvastatin | 0/15 | 15/15 | 0% | 3.6 ± 0.5 | 2.7% | 2.5 ± 0.6 | 7.4% | 12.4 ± 1.2 | 18.1 ± 2.1 |
| 10 mg/kg | | | | | | | p > 0.05 | | |
| BID | | | | | | | | | |
| Atorvastatin | 0/15 | 14/15 | 6.7% | 3.3 ± 1.0 | 10.8% | 2.3 ± 0.7 | 14.8% | 13.8 ± 4.9 | 17.2 ± 4.9 |
| 50 mg/kg | | | | | | | p > 0.05 | | |
| BID | | | | | | | | | |
| LAQUINIMOD | 0/15 | 14/15 | 6.7% | 2.7 ± 1.0 | 27.0% | 1.3 ± 0.6 | 51.9% | 15.1 ± 5.0 | 15.1 ± 4.9 |
| 5 mg/kg + | | | | | | | p < 0.001 | | |
| Atorvastatin | | | | | | | | | |
| 10 mg/kg | | | | | | | | | |
| LAQUINIMOD | 0/15 | 9/15 | 40% | 1.4 ± 1.5 | 62.2% | 0.7 ± 0.8 | 74.1% | 20.7 ± 8.8 | 8.1 ± 8.1 |
| 5 mg/kg + | | | | | p < 0.001 | | p < 0.001 | | |
| Atorvastatin | | | | | | | | | |
| 50 mg/kg | | | | | | | | | |

NA = not applicable.

### Example 2: Pharmacokinetics of Laquinimod and Atorvastatin in Plasma Following Oral Administrations of the Two Compounds Alone or Together in Male C57BL/6 Mice

The Purpose of this study is to determine PK profiles of Laquinimod and Atorvastatin in plasma after oral administrations of the two compounds alone or together in male C57BL/6 mice.

### Study Design

TABLE 4

| | | Dose | Dose | | |
|---|---|---|---|---|---|
| | | Level | Concentration | Dose | |
| Group | Treatment | (mg/kg) | (mg/mL) | Volume | Time Points |
| 1 | LAQ Sodium (PO) | 5 | 0.5 | 10 mL/kg | 0, 0.083, 0.25, 0.5, 1, |
| (n = 9) | | | | | 2, 4, 8 and 24 hr, |
| | | | | | plasma collection only |
| 2 | Atorvastatin Calcium | 50 | 5 | 10 mL/kg | 0, 0.083, 0.25, 0.5, 1, |
| (n = 9) | Trihydrate (PO) | | | | 2, 4, 8 and 24 hr, |
| | | | | | plasma collection only |
| 3 | Laquinimod Sodium & | 5 & 50 | 0.5 & 5 | 10 mL/kg | 0, 0.083, 0.25, 0.5, 1, |
| (n = 9) | Atorvastatin Calcium | | | | 2, 4, 8 and 24 hr, |
| | Trihydrate (PO) | | | | plasma collection only |

Test Article: Laquinimod Sodium, Atorvastatin Calcium Trihydrate

TABLE 5

| Compound ID | Laquinimod Sodium | | |
|---|---|---|---|
| MW (Free form) | 356.81 | FW (Salt form) | 378.79 |
| Purity | 99.8% | Appearance | White powder |
| Compound ID | Atorvastatin | | |
| | Calcium | | |
| | Trihydrate | | |
| MW (Free form) | 1117.3 | FW (Salt form) | 1209.44 |
| Purity | 95.3% | Appearance | White solid |

| Material | Manufacturer |
|---|---|
| MC (4633 mPa · s) | Fluka, Lot: 1388829 |

For all PO administration groups: 1% MC in water (all PO formulations were made just prior to use).

Preparation of PO Formulation for Laquinimod Alone (5 mg/kg, 10 mL/Kg) at 0.5 mg/mL
1) Weigh 2.08 mg of laquinimod sodium into a clean tube.
2) Add 3.919 mL of 1% MC in water to the tube containing the compound.
3) Stir the tube for 8-10 minutes and sonicate for 1-2 minutes.
   Preparation of PO Formulation for Atorvastatin Alone (50 mg/kg, 10 mL/kg) at 0.5 mg/mL
1) Weigh 17.42 mg of atorvastatin calcium trihydrate into a clean tube.
2) Add 3.067 mL of 1% MC in water to the tube containing the compound.
3) Stir the tube for 15-20 minutes and sonicate for 1-2 minutes.
   Preparation of PO Formulation for Laquinimod and Atorvastatin (5&50 mg/kg, 10 mL/kg) at (0.5 & 5) mg/mL
1) Weigh 2.04 mg of laquinimod sodium into a clean tube.
2) Add 3.843 mL of 1% MC in water to the tube containing the compound.
3) Stir the tube for 8-10 minutes and sonicate for 1-2 minutes.
4) Weigh 21.91 mg of atorvastatin calcium trihydrate into the tube.
5) Stir the tube for 15-20 minutes and sonicate for 1-2 minutes.

TABLE 6
Dosing solution analysis for Laquinimod by HPLC-UV; Dosing solution
concentration verification (Nominal concentration; 25 μg/mL)

| | Calculated | | | | |
|---|---|---|---|---|---|
| | conc. | Mean | | CV | Accuracy |
| Sample Name | (μg/mL) | (μg/mL) | SD | (%) | (%) |
| LAQ (PO-1) | 28.3 | 25.8 | 2.20 | 8.51 | 103 |
| LAQ (PO-2) | 25.1 | | | | |
| LAQ (PO-3) | 24.1 | | | | |
| LAQ + Atorvastatin | 23.4 | 22.8 | 0.742 | 3.25 | 91.2 |
| (PO-1) | | | | | |
| LAQ + Atorvastatin | 22.0 | | | | |
| (PO-2) | | | | | |
| LAQ + Atorvastatin | 23.1 | | | | |
| (PO-3) | | | | | |

PO doses of laquinimod were within 80%-120% of the theoretical concentration, so the nominal doses (5 mg/kg) were used for PK parameters estimation.

TABLE 7
Dosing solution analysis for atorvastatin by LC-MS/MS; Dosing solution
concentration verification (Nominal concentration: 100 ng/mL)

| | Calculated | | | | |
|---|---|---|---|---|---|
| | conc. | Mean | | CV | Accuracy |
| Sample Name | (ng/mL) | (ng/mL) | SD | (%) | (%) |
| atorvastatin (PO-1) | 97.4 | 95.2 | 4.55 | 4.78 | 5.2 |
| atorvastatin (PO-2) | 98.3 | | | | |
| atorvastatin (PO-3) | 90.0 | | | | |
| LAQ + Atorvastatin | 106 | 105 | 6.22 | 5.95 | 105 |
| (PO-1) | | | | | |
| LAQ + Atorvastatin | 97.8 | | | | |
| (PO-2) | | | | | |
| LAQ + Atorvastatin | 110 | | | | |
| (PO-3) | | | | | |

PO doses of atorvastatin were within 80%-120% of the theoretical concentration, so the nominal doses (50 mg/kg) were used for PK parameters estimation.

### Test System:

C57BL/6 mice, 17-18 g, male, N=27

### Administration:

PO Laquinimod: 5 mg/kg (10 mL/kg) via oral gavage (N=9)
PO Atorvastatin: 50 mg/kg (10 mL/kg) via oral gavage (N=9)
PO Laquinimod & Atorvastatin: 5 & 50 mg/kg (10 mL/kg) via oral gavage (N=9)

### Food Status:

Fasted overnight and fed 4 hr post dose, free access to water

### Analytical Method:

Instrument: LCMSMS-018 (API 5500)
Matrix: C57BL/6 mouse plasma
Analyte(s): Laquinimod and Atorvastatin
Internal standard(s): Dexamethasane
MS conditions: ESI: positive ion
   MRM detection
   Laquinimod: [M+H]⁺ m/z 357.0→122.2
   Atorvastatin: [M+H]⁺ m/z 559.3→440.4
   Dexamethasane: [M+H]⁺ m/z 393.1→373.1
HPLC conditions: Mobile phase:
   Mobile Phase A: H₂O—0.025% FA—1 mM NH₄OAc
   Mobile Phase B: MeOH—0.025% FA—1 mM NH₄OAc

| Time (min) | Mobile Phase B (%) |
|---|---|
| Initial | 10 |
| 0.20 | 10 |
| 0.80 | 80 |
| 1.10 | 80 |
| 1.11 | 95 |
| 1.20 | 95 |
| 1.21 | 10 |
| 1.80 | 10 |

Column: Waters BEH C18 (2.150 mm, 1.7 μm)
Flow rate: 0.60 mL/min
Column temperature: 60° C.
Retention time:
Laquinimod: 1.14 min; Atorvastatin: 1.19 min;
Dexamethasane: 1.09 min

### Sample Preparation:

1. For plasma samples: An aliquot of 30 μL plasma sample was added with 150 μL ACN which contains IS (Dexamethasane 40 ng/mL, 5 ng/mL) for protein precipitation. The mixture was vortexed for 2 rain and centrifuged at 12000 rpm for 5 min. The 2 μL supernatant was injected into LC-MS/MS.
2. For diluted plasma samples: An aliquot of 10 μL plasma sample was added with 90 μL blank plasma to obtain the diluted plasma samples, and the sample dilution factor is 10. An aliquot of 10 μL 10-folder-diluted plasma sample was added with 40 μL blank plasma to obtain the diluted plasma samples, and the sample dilution factor is 50. The exaction procedure for diluted samples was same as those for non-diluted samples.

### Calibration Curve:

1.00-3000 ng/mL for Laquinimod in mouse plasma
0.1-300 ng/mL for Atorvastatin in mouse plasma

### Blood Collection:

The animals were restrained manually at the designated time points. Approx. 150 μL of blood samples were taken from the animals into K2EDTA tubes via retro-orbital puncture or cardiac puncture for terminal bleeding under anesthesia with Isoflurane. Blood sample was put on ice and centrifuged at 2000 g for 5 min (4° C.) to obtain plasma sample within 15 minutes. Plasma samples were snap frozen by placing into dry-ice.

### Sample Storage and Disposition:

The plasma samples were stored at approximately −70° C. until analysis. The backup samples are discarded after two months unless specified. The unused dosing solutions are discarded within 1 week after completion of the study.

### Results

### PR Summary:

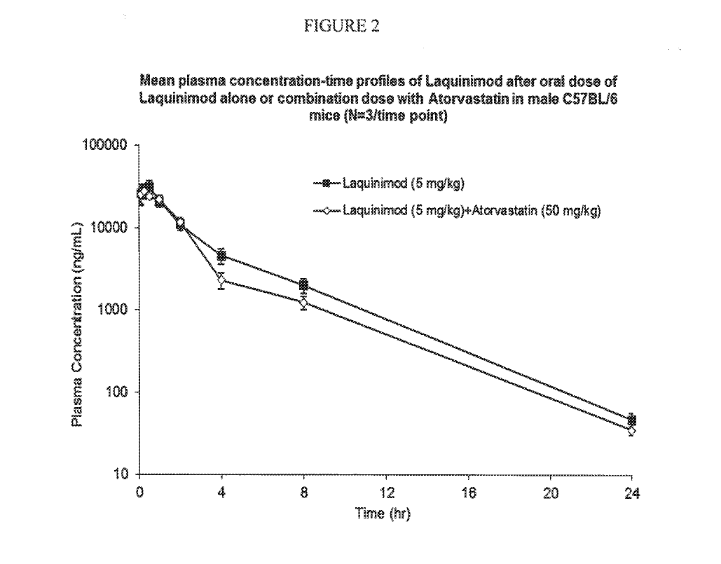

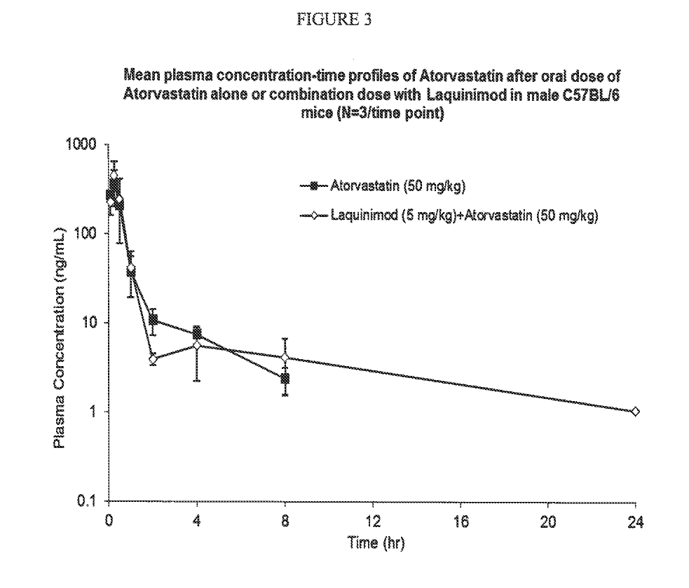

See Tables 8-11 below and FIGS. 2 and 3

TABLE 8
Individual and mean plasma concentration-time data of Laquinimod
after an oral dose at 5 mg/kg in male C57BL/6 mice

| | | Sampling | | | | | | |
|---|---|---|---|---|---|---|---|---|
| Dose | Dose | time | Individual Concentration | | | Mean | | CV |
| (mg/kg) | route | (hr) | (ng/mL) | | | (ng/mL) | SD | (%) |
| 5 | PO | 0 | BQL | BQL | BQL | BQL | NA | NA |
| | | 0.083 | 21300 | 34900 | 22100 | 26100 | 7632 | 29.2 |
| | | 0.25 | 30900 | 31800 | 21500 | 28067 | 5705 | 20.3 |
| | | 0.5 | 33900 | 35200 | 24200 | 31100 | 6011 | 19.3 |
| | | 1 | 17500 | 21600 | 23500 | 20867 | 3066 | 14.7 |
| | | 2 | 9060 | 11100 | 12500 | 10887 | 1730 | 15.9 |
| | | 4 | 3480 | 5380 | 4800 | 4553 | 974 | 21.4 |
| | | 8 | 1670 | 2450 | 1840 | 1987 | 410 | 20.6 |
| | | 24 | 37.0 | 46.3 | 57.1 | 46.8 | 10.1 | 21.5 |

| | |
|---|---|
| PK parameters | Unit |

| | | |
|---|---|---|
| Tₘₐₓ | hr | 0.500 |
| Cₘₐₓ | ng/mL | 31100 |
| Terminal t_{1/2} | hr | 3.01 |
| AUCₗₐₛₜ | hr*ng/mL | 86658 |
| AUC_{INF} | hr*ng/mL | 86861 |

TABLE 9
Individual and mean plasma concentration-time data of Atorvastatin
after an oral dose at 50 mg/kg in male C57BL/6 mice

| | | Sampling | | | | | | |
|---|---|---|---|---|---|---|---|---|
| Dose | Dose | time | Individual Concentration | | | Mean | | CV |
| (mg/kg) | route | (hr) | (ng/mL) | | | (ng/mL) | SD | (%) |
| 50 | PO | 0 | BQL | BQL | BQL | BQL | NA | NA |
| | | 0.083 | 235 | 301 | 272 | 269 | 33.1 | 12.3 |
| | | 0.25 | 540 | 238 | 306 | 361 | 158 | 43.8 |
| | | 0.5 | 186 | 229 | 207 | 207 | 21.5 | 10.4 |
| | | 1 | 26.0 | 59.0 | 28.3 | 37.8 | 18.4 | 48.8 |
| | | 2 | 6.83 | 11.9 | 13.6 | 10.8 | 3.52 | 32.7 |
| | | 4 | 8.53 | 8.37 | 5.49 | 7.46 | 1.71 | 22.9 |
| | | 8 | 2.75 | 1.47 | 2.91 | 2.38 | 0.789 | 33.2 |
| | | 24 | BQL | BQL | BQL | BQL | NA | NA |

| | |
|---|---|
| PK parameters | Unit |

| | | |
|---|---|---|
| Tₘₐₓ | hr | 0.250 |
| Cₘₐₓ | ng/mL | 361 |
| Terminal t_{1/2} | hr | 2.70 |
| AUCₗₐₛₜ | hr*ng/mL | 258 |
| AUC_{INF} | hr*ng/mL | 268 |

TABLE 10
Individual and mean plasma concentration-time data of Laquinimod after an oral
dose at 5 mg/kg Laquinimod and 50 mg/kg Atorvastatin in male C57BL/6 mice

| | | Sampling | | | | | | |
|---|---|---|---|---|---|---|---|---|
| Dose | Dose | time | Individual Concentration | | | Mean | | CV |
| (mg/kg) | route | (hr) | (ng/mL) | | | (ng/mL) | SD | (%) |
| 5 + 50 | PO | 0 | BQL | BQL | BQL | BQL | NA | NA |
| | | 0.083 | 23800 | 32200 | 20200 | 25400 | 6158 | 24.2 |
| | | 0.25 | 21900 | 30600 | 30000 | 27500 | 4859 | 17.7 |
| | | 0.5 | 24200 | 21900 | 25100 | 23733 | 1650 | 6.95 |
| | | 1 | 23600 | 23200 | 18600 | 21800 | 2778 | 12.7 |
| | | 2 | 11800 | 12900 | 10500 | 11733 | 1201 | 10.2 |
| | | 4 | 2840 | 1860 | 2190 | 2297 | 499 | 21.7 |
| | | 8 | 982 | 1320 | 1410 | 1237 | 226 | 18.2 |
| | | 24 | 37.6 | 29.8 | 37.4 | 34.9 | 4.45 | 12.7 |

| | |
|---|---|
| PK parameters | Unit |

| | | |
|---|---|---|
| Tₘₐₓ | hr | 0.250 |
| Cₘₐₓ | ng/mL | 27500 |
| Terminal t_{1/2} | hr | 3.25 |
| AUCₗₐₛₜ | hr*ng/mL | 71302 |
| AUC_{INF} | hr*ng/mL | 71465 |
| AUC_{Laq+AT}/AUC_{Laq} | | 0.823 |

TABLE 11
Individual and mean plasma concentration-time data of Atorvastatin after an oral
dose at 5 mg/kg Laquinimod and 50 mg/kg Atorvastatin in male C57BL/6 mice

| | | Sampling | | | | | | |
|---|---|---|---|---|---|---|---|---|
| Dose | Dose | time | Individual Concentration | | | Mean | | CV |
| (mg/kg) | route | (hr) | (ng/mL) | | | (ng/mL) | SD | (%) |
| 5 + 50 | PO | 0 | BQL | BQL | BQL | BQL | NA | NA |
| | | 0.083 | 190 | 300 | 189 | 226 | 63.8 | 28.2 |
| | | 0.25 | 547 | 576 | 219 | 447 | 198 | 44.3 |
| | | 0.5 | 99.7 | 208 | 430 | 246 | 168 | 68.5 |
| | | 1 | 38.8 | 64.8 | 20.6 | 41.4 | 22.2 | 53.7 |
| | | 2 | 4.59 | 3.55 | 3.65 | 3.93 | 0.574 | 14.6 |
| | | 4 | 9.44 | 3.33 | 3.97 | 5.58 | 3.36 | 60.2 |
| | | 8 | 1.78 | 6.92 | 3.71 | 4.14 | 2.60 | 62.8 |
| | | 24 | BQL | BQL | 1.05 | 1.05 | NA | NA |

| | |
|---|---|
| PK parameters | Unit |

| | | |
|---|---|---|
| Tₘₐₓ | hr | 0.250 |
| Cₘₐₓ | ng/mL | 447 |
| Terminal t_{1/2} | hr | 8.24 |
| AUCₗₐₛₜ | hr*ng/mL | 317 |
| AUC_{INF} | hr*ng/mL | 330 |
| AUC_{Laq+AT}/AUC_{AT} | | 1.23 |

1. No clinical findings were observed during the entire in-life study.
2. Dose levels mentioned above are of free acid.
3. BQL=Below quantifiable limit of 1.00 ng/mL for laquinimod, 0.1 ng/mL for atorvastatin in mouse plasma.
4. The oral dosing solutions were prepared in 1% MC in water.

### LC-MS MS:

See Tables 12-19

TABLE 12
SD curve of Laquinimod in mouse plasma

| | Anal. Conc. | Calculated Conc. | Accuracy |
|---|---|---|---|
| SD sample | (ng/mL) | (ng/mL) | (%) |
| STD1-01 | 1.00 | 1.03 | 103 |
| STD1-02 | 2.00 | 1.90 | 94.8 |
| STD1-03 | 10.0 | 8.83 | 88.3 |
| STD1-04 | 30.0 | 32.1 | 107 |
| STD1-05 | 100 | 88.4 | 88.4 |
| STD1-06 | 300 | 308 | 103 |
| STD1-07 | 1000 | 1020 | 102 |
| STD1-08 | 2700 | 2680 | 99.2 |
| STD1-09 | 3000 | 2860 | 95.2 |
| STD2-01 | 1.00 | 1.64* | NA |
| STD2-02 | 2.00 | 2.69* | NA |
| STD2-03 | 10.0 | 9.97 | 99.7 |
| STD2-04 | 30.0 | 33.2 | 111 |
| STD2-05 | 100 | 91.5 | 91.5 |
| STD2-06 | 300 | 313 | 104 |
| STD2-07 | 1000 | 1040 | 104 |
| STD2-08 | 2700 | 2710 | 101 |
| STD2-09 | 3000 | 3230 | 108 |

*The calculated value that was not within 85% to 115% (80% to 120% for LLOQ) of the theoretical value was excluded from calibration curve.

TABLE 13
SD curve of Laquinimod in mouse plasma for dilution

| | Anal. Conc. | Calculated Conc. | Accuracy |
|---|---|---|---|
| SD sample | (ng/mL) | (ng/mL) | (%) |
| STD1-01 | 1.00 | 1.12 | 112 |
| STD1-02 | 2.00 | 1.96 | 98.2 |
| STD1-03 | 10.0 | 8.54 | 85.4 |
| STD1-04 | 30.0 | 32.6 | 109 |
| STD1-05 | 100 | 99.3 | 99.3 |
| STD1-06 | 300 | 312 | 104 |
| STD1-07 | 1000 | 980 | 98.0 |
| STD1-08 | 2700 | 2710 | 100 |
| STD1-09 | 3000 | 3120 | 104 |
| STD2-01 | 1.00 | 0.972 | 97.2 |
| STD2-02 | 2.00 | 1.71 | 85.3 |
| STD2-03 | 10.0 | 8.27* | NA |
| STD2-04 | 30.0 | 28.9 | 96.5 |
| STD2-05 | 100 | 105 | 105 |
| STD2-06 | 300 | 315 | 105 |
| STD2-07 | 1000 | 1030 | 103 |
| STD2-08 | 2700 | 2550 | 94.6 |
| STD2-09 | 3000 | 3100 | 103 |

*The calculated value that was not within 85% to 115% (80% to 120% for LLOQ) of the theoretical value was excluded from calibration curve.

TABLE 14
QC samples of Laquinimod in mouse plasma

| QC | Anal. Conc. | Calculated Conc. | Accuracy |
|---|---|---|---|
| samples | (ng/mL) | (ng/mL) | (%) |
| QC-L | 3.00 | 3.05 | 102 |
| | 3.00 | 4.29* | NA |
| QC-M | 500 | 485 | 97.0 |
| | 500 | 494 | 98.7 |
| QC-H | 2400 | 2350 | 98.0 |
| | 2400 | 2470 | 103 |

*The calculated value was not within 85% to 115% of the theoretical value.

TABLE 15
QC samples of Laquinimod in mouse plasma for dilution

| QC | Anal. Conc. | Calculated Conc. | Accuracy |
|---|---|---|---|
| samples | (ng/mL) | (ng/mL) | (%) |
| QC-L | 3.00 | 3.31 | 110 |
| | 3.00 | 2.74 | 91.3 |
| QC-M | 500 | 510 | 102 |
| | 500 | 513 | 103 |
| QC-H | 2400 | 2470 | 103 |
| | 2400 | 2280 | 95.1 |
| DQC | 2400 | 2680 | 111 |
| | 2400 | 2950* | NA |
| | 2400 | 2640 | 110 |

*The calculated value was not within 85% to 115% of the theoretical value.

TABLE 16
SD curve of Atorvastatin in mouse plasma

| SD | Anal. Conc. | Calculated Conc. | Accuracy |
|---|---|---|---|
| sample | (ng/mL) | (ng/mL) | (%) |
| STD1-01 | 0.100 | 0.105 | 105 |
| STD1-02 | 0.200 | 0.192 | 95.8 |
| STD1-03 | 1.00 | 0.838* | NA |
| STD1-04 | 3.00 | 2.92 | 97.4 |
| STD1-05 | 10.0 | 9.15 | 91.5 |
| STD1-06 | 30.0 | 29.5 | 98.4 |
| STD1-07 | 100 | 105 | 105 |
| STD1-08 | 270 | 289 | 107 |
| STD1-09 | 300 | 341 | 114 |
| STD2-01 | 0.100 | 0.100 | 100 |
| STD2-02 | 0.200 | 0.188 | 94.1 |
| STD2-03 | 1.00 | 0.930 | 93.0 |
| STD2-04 | 3.00 | 3.02 | 101 |
| STD2-05 | 10.0 | 8.07* | NA |
| STD2-06 | 30.0 | 29.8 | 99.3 |
| STD2-07 | 100 | 96.6 | 96.6 |
| STD2-08 | 270 | 274 | 101 |
| STD2-09 | 300 | 371* | NA |

*The calculated value that was not within 85% to 115% (80% to 120% for LLOQ) of the theoretical vaiue was excluded from calibration curve.

TABLE 17
SD curve of Atorvastatin in mouse plasma for dilution

| SD | Anal. Conc. | Calculated Conc. | Accuracy |
|---|---|---|---|
| sample | (ng/mL) | (ng/mL) | (%) |
| STD1-01 | 0.100 | 0.110 | 110 |
| STD1-02 | 0.200 | 0.175 | 87.4 |
| STD1-03 | 1.00 | 0.632* | NA |
| STD1-04 | 3.00 | 2.70 | 90.0 |
| STD1-05 | 10.0 | 9.46 | 94.6 |
| STD1-06 | 30.0 | 33.6 | 112 |
| STD1-07 | 100 | 103 | 103 |
| STD1-08 | 270 | 290 | 108 |
| STD1-09 | 300 | 337 | 112 |
| STD2-01 | 0.100 | 0.104 | 104 |
| STD2-02 | 0.200 | 0.172 | 86.0 |
| STD2-03 | 1.00 | 0.672* | NA |
| STD2-04 | 3.00 | 2.49* | NA |
| STD2-05 | 10.0 | 8.67 | 86.7 |
| STD2-06 | 30.0 | 28.3 | 94.4 |
| STD2-07 | 100 | 103 | 103 |
| STD2-08 | 270 | 307 | 114 |
| STD2-09 | 300 | 289 | 96.4 |

*The calculated value that was not within 85% to 115% (80% to 120% for LLOQ) of the theoretical value was excluded from calibration curve.

TABLE 18
QC samples of Atorvastatin in mouse plasma

| QC | Anal. Conc. | Calculated Conc. | Accuracy |
|---|---|---|---|
| samples | (ng/mL) | (ng/mL) | (%) |
| QC-L | 0.300 | 0.255 | 85.1 |
| | 0.300 | 0.304 | 101 |
| QC-M | 50.0 | 55.7 | 111 |
| | 50.0 | 47.7 | 95.3 |
| QC-H | 240 | 251 | 104 |
| | 240 | 260 | 108 |

TABLE 19
QC samples of Atorvastatin in mouse plasma for dilution

| QC | Anal. Conc. | Calculated Conc. | Accuracy |
|---|---|---|---|
| samples | (ng/mL) | (ng/mL) | (%) |
| QC-L | 0.300 | 0.330 | 100 |
| | 0.300 | 0.360* | NA |
| QC-M | 50.00 | 44.3 | 88.7 |
| | 50.0 | 50.3 | 101 |
| QC-H | 240 | 231 | 96.1 |
| | 240 | 236 | 98.3 |
| DQC | 240 | 333* | NA |
| | 240 | 228 | 95.0 |
| | 240 | 206 | 85.9 |

*The calculated value was not within 85% to 115% of the theoretical value.

### Example 3: Assessment of Efficacy of Laquinimod as Add-on Therapy to Atorvastatin in Multiple Sclerosis (MS) Patients

Periodic oral administration of laquinimod as an add-on therapy for a human patient afflicted with a form of MS who is already receiving atorvastatin provides a clinically meaningful advantage and is more effective (provides at least an additive effect or more than an additive effect) in treating the patient than when atorvastatin is administered alone (at the same dose).

Periodic oral administration atorvastatin as an add-on therapy for a human patient afflicted with a form of MS who is already receiving of laquinimod provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment. As compared to when each agent is administered alone:
1. The add-on therapy is more effective (provides an additive effect or more than an additive effect) in sustaining (e.g., preventing, reducing or delaying) EDSS progression in multiple sclerosis patients after receiving the maintenance therapy for 6 months.
2. The add-on therapy is more effective (provides an additive effect or more than an additive effect) in reducing the decrease in brain volume (determined by the percent brain volume change (PBVC)), in multiple sclerosis patients.
3. The add-on therapy is more effective (provides an additive effect or more than an additive effect) in increasing the time to confirmed disease progression (CDP), in multiple sclerosis patients, where CDP is defined as a sustained increase in EDSS of ≧21 point from Baseline for at least 3 months. Progression cannot be confirmed during a relapse.
4. The add-on therapy is more effective (provides an additive effect or more than an additive effect) in reducing abnormalities observed in whole Brain MTR histogram, in multiple sclerosis patients.
5. The add-on therapy is more effective (provides an additive effect or more than an additive effect) in reducing the number of confirmed relapses and therefore the relapse rate, in multiple sclerosis patients.
6. The add-on therapy is also more effective (provides an additive effect or more than an additive effect) in reducing the accumulation of physical disability in multiple sclerosis patients, as measured by the time to confirmed progression of EDSS.
7. The add-on therapy is more effective (provides an additive effect or more than an additive effect) in reducing MRI-monitored disease activity in multiple sclerosis patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.
8. The add-on therapy is more effective (provides an additive effect or more than an additive effect) in reducing brain atrophy in multiple sclerosis patients.
9. The add-on therapy is more effective (provides an additive effect or more than an additive effect) in reducing the frequency of relapses, the frequency of clinical exacerbation, and the risk for confirmed progression in multiple sclerosis patients.
10. The add-on therapy is more effective (provides an additive effect or more than an additive effect) in increasing the time to confirmed relapse in multiple sclerosis patients.
11. The add-on therapy is more effective (provides an additive effect or more than an additive effect) in improving the general health status (as assessed by the EuroQoL (EQ5D) questionnaire), symptom severity on work (as assessed by the work productivity and activities impairment General Health (WPAI-GH) questionnaire) and quality of life, in multiple sclerosis patients.
12. The add-on therapy is more effective (provides an additive effect or more than an additive effect) in decreasing cerebral dysfunction/cognitive impairment (as assessed by Symbol Digit Modalities Test (SDHT)), in multiple sclerosis patients during the double blind study period.

### Example 4: Assessment of Efficacy of Laquinimod as Add-on Therapy to Atorvastatin in CIS Patients

Administration of laquinimod as an add-on therapy to atorvastatin provides a clinically meaningful advantage and is more effective (provides an additive effect or more than an additive effect) in delaying the conversion to clinically definite MS in patients presenting a CIS suggestive of MS than when atorvastatin is administered alone (at the same dose).

Administration of laquinimod as an add-on therapy to atorvastatin provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when atorvastatin is administered alone (at the same dose).

Administration of atorvastatin as an add-on therapy to laquinimod provides a clinically meaningful advantage and is more effective (provides an additive effect or more than an additive effect) in delaying the conversion to clinically definite MS in patients presenting a CIS suggestive of MS than when laquinimod is administered alone (at the same dose).

Administration of atorvastatin as an add-on therapy to laquinimod provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when laquinimod is administered alone (at the same dose).

### Example 5: Assessment of Efficacy of Laquinimod in Combination with Atorvastatin in Multiple Sclerosis (MS) Patients

Periodic oral administration of laquinimod in combination with atorvastatin to a human patient afflicted with relapsing form of multiple sclerosis provides increased efficacy (provides at least an additive effect or more than an additive effect) in treating the patient than when laquinimod is administered alone or when atorvastatin is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or atorvastatin is administered alone (at the same dose) in the following manner:
1. The combination therapy is more effective (provides an additive effect or more than an additive effect) in sustaining (e.g., preventing, reducing or delaying) EDSS progression in multiple sclerosis patients after receiving the maintenance therapy for 6 months.
2. The combination therapy is more effective (provides an additive effect or more than an additive effect) in reducing the decrease in brain volume (determined by the percent brain volume change (PBVC)), in multiple sclerosis patients.
3. The combination therapy is more effective (provides an additive effect or more than an additive effect) in increasing the time to confirmed disease progression (CDP), in multiple sclerosis patients, where CDP is defined as a sustained increase in EDSS of ≧1 point from Baseline for at least 3 months. Progression cannot be confirmed during a relapse.
4. The combination therapy is more effective (provides an additive effect or more than an additive effect) in reducing abnormalities observed in whole Brain MTR histogram, in multiple sclerosis patients during.
5. The combination therapy is more effective (provides an additive effect or more than an additive effect) in reducing the number of confirmed relapses and therefore the relapse rate, in multiple sclerosis patients.
6. The combination therapy is also more effective (provides an additive effect or more than an additive effect) in reducing the accumulation of physical disability in multiple sclerosis patients, as measured by the time to confirmed progression of EDSS.
7. The combination therapy is more effective (provides an additive effect or more than an additive effect) in reducing MRI-monitored disease activity in multiple sclerosis patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.
8. The combination therapy is more effective (provides an additive effect or more than an additive effect) in reducing brain atrophy in multiple sclerosis patients.
9. The combination therapy is more effective (provides an additive effect or more than an additive effect) in reducing the frequency of relapses, the frequency of clinical exacerbation, and the risk for confirmed progression in multiple sclerosis patients.
10. The combination therapy is more effective (provides an additive effect or more than an additive effect) in increasing the time to confirmed relapse in multiple sclerosis patients.
11. The combination therapy is more effective (provides an additive effect or more than an additive effect) in improving the general health status (as assessed by the EuroQoL (EQ5D) questionnaire), symptom severity on work (as assessed by the work productivity and activities impairment General Health (WPAI-GH) questionnaire) and quality of life, in multiple sclerosis patients.
12. The combination therapy is more effective (provides an additive effect or more than an additive effect) in decreasing cerebral dysfunction/cognitive impairment (as assessed by Symbol Digit Modalities Test (SDMT)), in multiple sclerosis patients during the double blind study period.

### Example 6: Assessment of Efficacy of Laquinimod in Combination with Atorvastatin Therapy in CIS Patients

Administration of laquinimod in combination with atorvastatin provides a clinically meaningful advantage and is more effective (provides an additive effect or more than an additive effect) in delaying the conversion to clinically definite MS in patients presenting a CIS suggestive of MS than when atorvastatin is administered alone (at the same dose).

Administration of laquinimod in combination with atorvastatin provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when atorvastatin is administered alone (at the same dose).

### Example 7: Assessment of Efficacy of Suboptimal Doses of Laquinimod in Combination with Suboptimal Doses of Atorvastatin in Multiple Sclerosis (MS) Patients

Periodic oral administration of suboptimal dose of laquinimod as an add-on to suboptimal dose of atorvastatin to a human patient afflicted with relapsing form of multiple sclerosis is as least as effective or more effective in treating the patient than when laquinimod is administered alone or when atorvastatin is administered alone (at the respective optimal doses). The add-on therapy also provides efficacy in treating the patient without undue adverse side effects or affecting the safety of the treatment.

Periodic oral administration of suboptimal dose of atorvastatin as an add-on to suboptimal dose of laquinimod to a human patient afflicted with relapsing form of multiple sclerosis is as least as effective or more effective in treating the patient than when laquinimod is administered alone or when atorvastatin is administered alone (at the respective optimal doses). The add-on therapy also provides efficacy in treating the patient without undue adverse side effects or affecting the safety of the treatment.

Periodic oral administration of suboptimal dose of atorvastatin in combination with suboptimal dose of laquinimod to a human patient afflicted with relapsing form of multiple sclerosis is as least as effective or more effective in treating the patient than when laquinimod is administered alone or when atorvastatin is administered alone (at the respective optimal doses). The combination therapy also provides efficacy in treating the patient without undue adverse side effects or affecting the safety of the treatment.

Laquinimod and atorvastatin add-on therapy provides advantages as compared therapy using individual agent alone including improved relapse rate reduction, improved preservation of brain tissue, improved reduction in disability progression and improved safety profile, with reduced respective doses.

Laquinimod and atorvastatin combination therapy provides advantages as compared therapy using individual agent alone including improved relapse rate reduction, improved preservation of brain tissue, improved reduction in disability progression and improved safety profile, with reduced respective doses.

### REFERENCES

1. Armen at al. Principles of Pharmacology: The Pathophysiologic Basis of Drug Therapy (3rd ed.), U.S.: Lippincott Williams & Wilkins, 2011.
2. Barkhof, F. (1999) “MRI in Multiple Sclerosis: Correlation with Expanded Disability Status Scale (EDSS)”, Multiple Sclerosis. 5(4):283-286 (Abstract).
3. Bjartmar C and Fox R I. (2002) “Pathological mechanisms and disease progression of multiple sclerosis: therapeutic implication”, Drugs of Today. 38:7-29.
4. Brex et al., (2002) “A longitudinal study of abnormalities on MRI and disability from multiple sclerosis”, N Engl J Med. Jan. 17, 2002 346(3):158-64.
5. Brod at al. (2000) Annals of Neurology, 47:127-131.
6. Brück (2011) “Insight into the mechanism of laquinimod action.” J Neurol Sci. 2011 Jul. 15; 306(1-2):173-9.
7. Brunmark C at al., (2002) “The new orally active immunoregulator laquinimod (ABR-215062) effectively inhibits development and relapses of experimental autoimmune encephalomyelitis”, J Neuroimmunology. 130:163-172.

B. Comi at al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology; April 28-May 5, 2007; Boston, Mass.
9. Compston, Genetic susceptibility to multiple sclerosis, in McAlpine's Mutiple Sclerosis, Matthews, B. ed., London: Churchill Livingstone, 1991, 301-319.
10. Conway and Cohen (2010) “Combination therapy in multiple sclerosis”, LancetNeurol, 9:299-308.
11. Costello et al. (2007) “Combination therapies for multiple sclerosis: scientific rationale, clinical trials, and clinical practice”, Current Opinion in Neurology, 20:281-285.
12. Dal Canto et al. (1977) Multiple sclerosis. Animal model: Theiler's virus infection in mice. Am. J. Path. 88:497-500.
13. De Stefano at al. (1999) “Evidence of early axonal damage in patients with multiple sclerosis”, Neurology. 52(Suppl 2):A378.
14. Dunitz, M. Multiple sclerosis therapeutics, Ed. Rudick and Goodkin. London: Taylor & Francis, 1999.
15. EMEA Guideline on Clinical Investigation of Medicinal Products for the Treatment of Multiple Sclerosis (CPMP/EWP/561/98 Rev. 1, Nove.2006).
16. Fernández (2007) “Combination therapy in multiple sclerosis”, Journal of the neurological sciences, 259:95-103.
17. Fischer et al., (1999) “The Multiple Sclerosis Functional Composite measure (MSFC): an integrated approach to MS clinical outcome assessment” Multiple Sclerosis, 5(4):244-250.
18. Fisk et al., (1994) “Measuring the Functional Impact of Fatigue: Initial Validation of Fatigue Impact Scale”, Clin Inf Dis. 18 Suppl 1:S79-83.
19. Fisk et al., (1994) “The Impact of Fatigue on Patients with Multiple Sclerosis”, Can J Neurol Sci. 21:9-14.
20. Frenández (2007) “Combination therapy in multiple sclerosis”, Journal of the neurological sciences, 259:95-103.
21. Frohman et al., (2003) “The utility of MRI in suspected MS: report of the Therapeutics and Technology Assessment Subcommittee of the American Academy of Neurology”, Neurology. Sep. 9, 2003, 61(5):602-11.
22. Gold (2008) “Combination therapies in multiple sclerosis”, J Neurol, 255(Suppl 11:51-60.
23. Golder W, (2007) “Magnetic resonance spectroscopy in clinical oncology”, Onkologie. 27(3): 304-9.
24. Grossman et al., (1994) Magnetization transfer: theory and clinical applications in neuroradiology”, RadioGraphics. 14:279-290.
25. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999 <http://www.fda.gov/cber/gdlns/metabol.pdf>.
26. Guidance for Industry. Drug Interaction Studies—Study Design, Data Analysis, Implications for Dosing, and Labeling Recommendations, U.S. Dept. Health and Human Svcs., FDA, Ctr. for Drug Eval. and Res. (CDER), February 2012, Clinical Pharmacology.
27. Gurevich et al. (2010) “Laquinimod suppress antigen presentation in relapsing-remitting multiple sclerosis: in vitro high-throughput gene expression study” (J Neuroimmunol. 2010 Apr. 15; 221(1-2):87-94. Epub 2010 Mar. 27.
28. Guyatt et al. (1985) “The 6-minute walk: a new measure of exercise capacity in patients with chronic heart failure”, Can Med Assoc J., 132:919-823.
29. Hafler and Weiner, M S: A CNS and systemic autoimmune disease, Immunol. Today, 1989, 10:104-107.
30. Hartung at al. (2005) “Significance of neutralizing antibodies to interferon beta during treatment of multiple sclerosis: expert opinions based on the Proceedings of an International Consensus Conference”, Eur J Neurol. 12:588-601.
31. Hauser et al. (1983) “Intensive immunosuppression in progressive multiple sclerosis”, New Engl J Med. 308:173-180.
32. Hohlfeld et al. (2000) “The neuroprotective effect of inflammation: implications for the therapy of multiple sclerosis”, J Neuroimmunol. 107:161-166.
33. Johnson et al. (1986) “Cell-mediated immunity to myelin-associated glycoprotein, proteolipid protein, and myelin basic protein in multiple sclerosis”, J Neuroimmunol. 1986 November; 13(1):99-108.
34. Kleinschmidt-DeMasters et al. (2005) New England Journal of Medicine, 353:369-379.
35. Kurtzke J F, (1983) “Rating neurologic impairment in multiple sclerosis: an expanded disability status scale (EDSS)”, Neurology 33(11):1444-1452.
36. Lampert, Autoimmune and virus-induced demyelinating diseases. A review, Am. J. Path., 1978, 91:176-208.
37. Langer-Gould at al. (2005) New England Journal of Medicine, 353:369-379.
38. Lublin F D, Reingold S C (1996) “Defining the clinical course of multiple sclerosis”, Neurol. 46:907-911.
39. Martyn, The epidemiology of multiple sclerosis, in McAlpine's Multiple Sclerosis, Matthews, B., ed., London: Churchil Livingstone, 1991, 3-40.
40. McDonald et al., Recommended diagnostic criteria for multiple sclerosis: guidelines from the International Panel on the diagnosis of multiple sclerosis. Ann. Neurol., 2001, 50:121-127.
41. McDonald, (2001) “Guidelines from the International Panel on the Diagnosis of Multiple Sclerosis” Ann. Neurol. 50:121-127.
42. Mehta et al. (1996) “Magnetization transfer magnetic resonance imaging: a clinical review”, Topics in Magnetic Resonance Imaging 8(4):214-30.
43. Miki et al. (1999) “Relapsing-Remitting Multiple Sclerosis: Longitudinal Analysis of MR Images—Lack of Correlation between Changes in T2 Lesion Volume and Clinical Findings”, Radiology. 213:395-399.
44. Milo and Panitch (2011) “Combination therapy in multiple sclerosis”, Journal of Neuroimmunology, 231(2011):23-31.
45. Moraal et al. (2008) “Subtraction MR Images in a Multiple Sclerosis Multicenter Clinical Trial Setting”, Radiology, 250(2):506-514.
46. Multiple sclerosis: its diagnosis, symptoms, types and stages, 2003<http://www.albany.net/˜tjc/multiple-sclerosis.html>.
47. National MS Society Website, retrieved Jul. 10, 2012<http://www.nationalmssociety.org/ms-clinical-care-network/researchers/clinical-study-measures/index.aspx>
48. Neuhaus et al. (2003) “Immunomodulation in multiple sclerosis: from immunosuppression to neuroprotection”, Trends Pharmacol Sci. 24:131-138.
49. Noseworthy et al. (2000) “Multiple sclerosis”, N Engl J Med. 343:938-952.
50. Olsson, Immunology of multiple sclerosis, Curr. Opin. Neurol. Neurosurg., 1992, 5:195-202.
51. PCT International Application Publication No. WO 1998/30227, published Jul. 16, 1998.
52. PCT International Application Publication No. WO 2000/05250, published Feb. 3, 2000.
53. PCT International Application Publication No. WO 2000/18794, published Apr. 6, 2000.
54. PCT International Application Publication No. WO 2003/048735, published Jun. 12, 2003.
55. PCT International Application Publication No. WO 2004/103297, published Dec. 2, 2004.
56. PCT International Application Publication No. WO 2004/112754, published Dec. 29, 2004.
57. PCT International Application Publication No. WO 2006/016036, published Nov. 2, 2006.
58. PCT International Application Publication No. WO 2006/029393, published Mar. 16, 2006.
59. PCT International Application Publication No. WO 2006/029411, published Mar. 16, 2006.
60. PCT International Application Publication No. WO 2006/083608, published Aug. 10, 2006.
61. PCT International Application Publication No. WO 2006/089164, published Aug. 24, 2006.
62. PCT International Application Publication No. WO 2006/116602, published Nov. 2, 2006.
63. PCT International Application Publication No. WO 2007/047863, published Apr. 26, 2007.
64. PCT International Application Publication No. WO 2007/146248, published Dec. 21, 2007, international filing date Jun. 12, 2007.
65. PCT International Application Publication No. WO 2009/070298, published Jun. 4, 2009.
66. PCT International Application Publication No. WO 2011/008274, published Jan. 20, 2011.
67. PCT International Application Publication No. WO 2011/022063, published Feb. 24, 2011.
68. PCT International Application Publication No. WO 2011/157719, published Dec. 22, 2011.
69. PCT International Application Publication No. WO 2012/051106, published Apr. 19, 2012.
70. Physicians' Desk Reference Website, retrieved Nov. 6, 2014 <http://www.pdr.net/drug-summary/lipitor?druglabelid-2338>
71. Polman at al. (2011) “Diagnostic Criteria for Multiple Sclerosis: 2010 Revisions to the McDonald Criteria”, Ann Neural, 69:292-302.
72. Polman et al., (2005) “Diagnostic criteria for multiple sclerosis: 2005 revisions to the McDonald Criteria”, Annals of Neurology, Volume 58 Issue 6, Pages 840-846.
73. Polman et al., (2005) “Treatment with laquinimod reduces development of active MRI lesions in relapsing MS”, Neurology. 64:987-991.
74. Poser et al. (1983) “New Diagnostic Criteria for Multiple Sclerosis: Guidelines for Research Protocols”, Annals of Neurology, March 1983, 13(3):227-230.
75. Postmus et al. (2014) “Pharmacogenetic meta-analysis of genome-wide association studies of LDL cholesterol response to statins”, Nature Communications, October 2014, 5:5068.
76. Rosen Y, (2007) “The Recent advances in magnetic resonance neurospectroscopy”, Neurotherapeutics. 27(3):330-45.
77. RTT News Article dated Apr. 12, 11, entitled “Teva Pharma, Active Biotech Post Positive Laquinimod Phase 3 ALLEGRO Results”.
78. Rudick at al. (2006) New England Journal of Medicine, 354:911-923.
79. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.
80. Runström at al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
81. Runström at al. (2006) “Inhibition of the development of chronic experimental autoimune encephalomyelitis by laquinimod (ABR-215062) in IFN-β k.o. and wild type mice” Journal of Neuroimmunology, 173(2006):69-78.
82. Salama at al. (2003) Multiple Sclerosis, 9:28-31.
83. Sandberg-Wollheim at al. (2005) “48-week open safety study with high-dose oral laquinimod in patients”, Mult Scler. 11:S154 (Abstract).
84. Teva Press Release dated Aug. 1, 2011, entitled “Results of Phase III BRAVO Trial Reinforce Unique Profile of Laquinimod for Multiple Sclerosis Treatment”.
85. The National MS Society (USA), The Disease Modifying Drug Brochure, Oct. 19, 2006.
86. U.S. Food and Drug Administration Website, retrieved Nov. 6, 2014 <http://www.fda.gov/Drugs/DrugSafety/InformationbyDrugClass/ucm 294358.htm>
87. U.S. Patent Application Publication No. 2008-0279930, published Nov. 13, 2008 (Terhaag at al.).
88. U.S. Patent Application Publication No. 2008-0207526, published Aug. 28, 2008 (Strominger et al.).
89. U.S. Patent Application Publication No. 2008-0279952, published Nov. 13, 2008 (Bernd Terhaag and Pirna).
90. U.S. Patent Application Publication No. 2010-0322900, published Dec. 23, 2010 (Nora Tarcic and Modiin).
91. U.S. Patent Application Publication No. 2011-0027219, published Feb. 3, 2011 (Tarcic et al.).
92. U.S. Patent Application Publication No. 2011-0034508, published Feb. 10, 2011 (Liat Hayardeny).
93. U.S. Patent Application Publication No. 2011-0217295, published Sep. 8, 2011 (Haviv and Tarcic).
94. U.S. Patent Application Publication No. 2011-0218179, published Sep. 8, 2011 (Haviv and Tarcic).
95. U.S. Patent Application Publication No. 2011-0218203, published Sep. 8, 2011 (Joel Kaye et al.).
96. U.S. Patent Application Publication No. 2011-0230413, published Sep. 22, 2011 (Suhayl Dhib-Jalbut).
97. U.S. Patent Application Publication No. 2012-0010238, published Jan. 12, 2012 (Fristedt).
98. U.S. Patent Application Publication No. 2012-0010239, published Jan. 12, 2012 (Piryatinsky et al.).
99. U.S. Patent Application Publication No. 2012-0142730, published Jun. 7, 2012 (Tarcic et al.).
100. U.S. Pat. No. 3,849,550, issued Nov. 19, 1974 (Teitelbaum et al).
101. U.S. Pat. No. 5,284,861, issued Feb. 8, 1994 (Peter Emig et al.).
102. U.S. Pat. No. 5,721,258, issued Feb. 24, 1998 (Jurgen Engel et al.).
103. U.S. Pat. No. 5,800,808, issued Sep. 1, 1998 (Konfino et al).
104. U.S. Pat. No. 5,858,964, issued Jan. 12, 1999 (Aharoni et al).
105. U.S. Pat. No. 5,981,589, issued Nov. 9, 1999 (Konfino et al).
106. U.S. Pat. No. 6,048,898, issued Apr. 11, 2000 (Konfino at al).
107. U.S. Pat. No. 6,054,430, issued Apr. 25, 2000 (Konfino et al).
108. U.S. Pat. No. 6,077,851, issued Jun. 20, 2000 (Bjork et al).
109. U.S. Pat. No. 6,194,000, issued Feb. 27, 2001 (Smith et al.).
110. U.S. Pat. No. 6,214,791, issued Apr. 10, 2001 (Arnon et al).
111. U.S. Pat. No. 6,342,476, issued Jan. 29, 2002 (Konfino et al).
112. U.S. Pat. No. 6,362,161, issued Mar. 26, 2002 (Konfino et al).
113. U.S. Pat. No. 7,566,767, issued Jul. 28, 2009 (Strominger et al.).
114. U.S. Pat. No. 7,589,208, issued Sep. 15, 2009 (Jansson et al).
115. U.S. Pat. No. 7,884,208, issued Feb. 8, 2011 (Frenkel et al.).
116. U.S. Pat. No. 7,989,473, issued Aug. 2, 2011 (Patashnik et al.).
117. U.S. Pat. No. 8,008,258, issued Aug. 30, 2011 (Aharoni et al).
118. U.S. Pat. No. 8,178,127, issued May 15, 2012 (Safadi et al.).
119. Vollmer et al. (2008) “Glatiramer acetate after induction therapy with mitoxantrone in relapsing multiple sclerosis” Multiple Sclerosis, 00:1-8.
120. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
121. Yong (2002) “Differential mechanisms of action of interferon-n and glatiramer acetate in MS” Neurology, 59:1-7.
122. Zou et al. (2002) “Suppression of experimental autoimmune neuritis by ABR-215062 is associated with altered Th1/Th2 balance and inhibited migration of inflammatory cells into the peripheral nerve tissue”, Neuropharmacology. 42:731.

What is claimed is:

1. A method of treating a subject afflicted with multiple sclerosis (MS) or presenting a clinically isolated syndrome (CIS) comprising administering to the subject an amount of laquinimod and administering to the subject an amount of a statin.

2. The method of claim 1, wherein the amount of laquinimod and the amount of the statin when taken together is more effective to treat the subject than when each agent at the same respective amount is administered alone.

3. The method of any one of claims 1 or 2, wherein the MS is relapsing MS.

4. The method of claim 3, wherein the relapsing MS is relapsing-remitting MS.

5. The method of any one of claims 1-4, wherein the amount of laquinimod and the amount of the statin when taken together is effective to reduce a symptom of MS in the subject.

6. The method of claim 5, wherein the symptom is a MRI-monitored MS disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

7. The method of claim 6, wherein the amount of laquinimod and the amount of the statin when taken together is effective to
a) decrease or inhibit reduction of brain volume,
b) increase time to confirmed disease progression,
c) decrease abnormalities observed in whole Brain MTR histogram, or
d) reduce cognitive impairment.

8. The method of claim 7, wherein brain volume is measured by percent brain volume change (PBVC).

9. The method of claim 7, wherein time to confirmed disease progression is increased by 20-60%.

10. The method of claim 7, wherein cognitive impairment is assessed by the Symbol Digit Modalities Test (SDMT) score.

11. The method of claim 6, wherein the accumulation of physical disability is measured by Kurtzke Expanded Disability Status Scale (EDSS) score, or is assessed by the time to confirmed disease progression as measured by EDSS score.

12. The method of claim 6, wherein the subject had an EDSS score of 0-5.5 at baseline, an EDSS score of 1.5-4.5 at baseline or an EDSS score of 5.5 or greater at baseline.

13. The method of claims 11 or 12, wherein confirmed disease progression is a 1 point or a 0.5 point increase of the EDSS score.

14. The method of claim 6, wherein impaired mobility is assessed by the Timed-25 Foot Walk test, the 12-Item MS Walking Scale (MSWS-12) self-report questionnaire, the Ambulation Index (AI), the Six-Minute Walk (6MW) Test or the Lower Extremity Manual Muscle Test (LEMMT) Test.

15. The method of claim 6, wherein general health status is assessed by the EuroQoL (EQ5D) questionnaire, Subject Global Impression (SGI) or Clinician Global Impression of Change (CGIC).

16. The method of claim 6, wherein functional status is measured by the subject's Short-Form General Health survey (SF-36) Subject Reported Questionnaire score.

17. The method of claim 6, wherein quality of life is assessed by SF-36, EQ5D, Subject Global Impression (SGI) or Clinician Global Impression of Change (CGIC).

18. The method of claims 16 or 17, wherein the subject's SF-36 mental component summary score (MSC) is improved.

19. The method of any one of claims 16-18, wherein the subject's SF-36 physical component summary sore (PSC) is improved.

20. The method of claim 6, wherein fatigue is assessed by the EQ5D, the subject's Modified Fatigue Impact Scale (MFIS) score or the French valid versions of the Fatigue Impact Scale (EMIF-SEP) score.

21. The method of claim 6, wherein symptom severity on work is measured by the work productivity and activities impairment General Health (WPAI-GH) questionnaire.

22. The method of any one of claims 1-21, wherein laquinimod is laquinimod sodium and/or the statin is atorvastatin calcium.

23. The method of any one of claims 1-22, wherein the laquinimod and/or the statin is administered via oral administration.

24. The method of any one of claims 1-23, wherein the laquinimod and/or the statin is administered periodically.

25. The method of any one of claims 1-24, wherein the laquinimod and/or the statin is administered daily.

26. The method of any one of claims 1-24, wherein the laquinimod and/or the statin is administered more often than once daily or less often than once daily.

27. The method of any one of claims 1-26, wherein the amount of laquinimod administered is less than 0.6 mg/day.

28. The method of any one of claims 1-26, wherein the amount of laquinimod administered is 0.1-40.0 mg/day.

29. The method of claim 28, wherein the amount of laquinimod administered is 0.1-2.5 mg/day.

30. The method of claim 28, wherein the amount of laquinimod administered is 0.25-2.0 mg/day.

31. The method of claim 28, wherein the amount of laquinimod administered is 0.5-1.2 mg/day.

32. The method of claim 28, wherein the amount of laquinimod administered is 0.25 mg/day, 0.3 mg/day, 0.5 mg/day, 0.6 mg/day, 1.0 mg/day, 1.2 mg/day, 1.5 mg/day or 2.0 mg/day.

33. The method of any one of claims 1-32, wherein the amount of the statin administered is 0.1-100 mg/day.

34. The method of claim 33, wherein the amount of the statin administered is 10-80 mg/day.

35. The method of claim 34, wherein the amount of statin administered is about 10, 20, 40, or 80 mg/day.

36. The method of claim 35, wherein the amount of the statin administered is 10, 20, 40, or 80 mg/day.

37. The method of any one of claims 24-36, wherein a loading dose of an amount different from the intended dose is administered for a period of time at the start of the periodic administration.

38. The method of any one of claims 1-37, wherein the subject is receiving laquinimod therapy prior to initiating the statin therapy.

39. The method of any one of claims 1-37, wherein the subject is receiving the statin therapy prior to initiating laquinimod therapy.

40. The method of claims 38 or 39, where in the subject is receiving a first therapy for at least 8 weeks, at least 10 weeks, at least 24 weeks, at least 28 weeks, at least 48 weeks or at least 52 weeks prior to initiating a second therapy.

41. The method of any one of claims 1-40, further comprising administration of nonsteroidal anti-inflammatory drugs (NSAIDs), salicylates, slow-acting drugs, gold compounds, hydroxychloroquine, sulfasalazine, corticosteroids, cytotoxic drugs, immunosuppressive drugs and/or antibodies.

42. The method of any one of claims 24-41, wherein the periodic administration of laquinimod and/or the periodic administration of the statin continues for at least 3 days, for more than 30 days, for more than 42 days, for 8 weeks or more, for at least 12 weeks, for at least 24 weeks or for 6 months or more.

43. The method of any one of claims 1-42, wherein the administration of laquinimod and the administration of the statin inhibits a symptom of relapsing MS by at least 20%, by at least 30%, by at least 50%, by at least 70%, by more than 100%, by more than 300% or by more than 1000%.

44. The method of any one of claims 1-43, wherein each of the amount of laquinimod when taken alone, and the amount of the statin when taken alone is effective to treat the subject.

45. The method of any one of claims 1-43, wherein either the amount of laquinimod when taken alone, the amount of the statin when taken alone, or each such amount when taken alone is not effective to treat the subject.

46. The method of any one of claims 1-45, wherein the subject is a human patient.

47. A package comprising:
a) a first pharmaceutical composition comprising an amount of laquinimod and a pharmaceutically acceptable carrier;
b) a second pharmaceutical composition comprising an amount of a statin and a pharmaceutically acceptable carrier; and
c) instructions for use of the first and second pharmaceutical compositions together to treat a subject afflicted with MS or presenting a CIS.

48. The package of claim 47, wherein the first pharmaceutical composition, the second pharmaceutical composition, or both the first and the second pharmaceutical compositions are in an aerosol, an inhalable powder, an injectable, a liquid, a solid, a capsule or a tablet form.

49. The package of claim 48, wherein the first pharmaceutical composition, the second pharmaceutical composition, or both the first and the second pharmaceutical compositions are in a liquid or a solid form.

50. The package of claim 49, wherein the first pharmaceutical composition, the second pharmaceutical composition, or both the first and the second pharmaceutical compositions are in capsule form or in tablet form.

51. The package of claim 50, wherein the tablets are coated with a coating which inhibits oxygen from contacting the core.

52. The package of claim 51, wherein the coating comprises a cellulosic polymer, a detackifier, a gloss enhancer, or pigment.

53. The package of any one of claims 47-52, wherein the first pharmaceutical composition further comprises mannitol, an alkalinizing agent, an oxidation reducing agent, a lubricant, and/or a filler.

54. The package of claim 53, wherein the alkalinizing agent is meglumine.

55. The package of claims 53 or 54, wherein the lubricant is present in the composition as solid particles.

56. The package of any one of claims 53-55, wherein the lubricant is sodium stearyl fumarate or magnesium stearate.

57. The package of any one of claims 53-56, wherein the filler is present in the composition as solid particles.

58. The package of any one of claims 53-57, wherein the filler is lactose, lactose monohydrate, starch, isomalt, mannitol, sodium starch glycolate, sorbitol, lactose spray dried, lactose anhydrouse, or a combination thereof.

59. The package of claim 58, wherein the filler is mannitol or lactose monohydrate.

60. The package of any one of claims 47-59, wherein the first pharmaceutical composition is stable and free of an alkalinizing agent or an oxidation reducing agent.

61. The package of claim 60, wherein the first pharmaceutical composition is free of an alkalinizing agent and free of an oxidation reducing agent.

62. The package of any one of claims 47-61, wherein the first pharmaceutical composition is stable and free of disintegrant.

63. The package of any one of claims 47-62, further comprising a desiccant.

64. The package of claim 63, wherein the desiccant is silica gel.

65. The package of any one of claims 47-64, wherein the first pharmaceutical composition is stable and has a moisture content of no more than 4%.

66. The package of any one of claims 47-65, wherein laquinimod is present in the composition as solid particles.

67. The package of any one of claims 47-66, wherein the package is a sealed packaging having a moisture permeability of not more than 15 mg/day per liter.

68. The package of any one of claim 53-67, wherein the sealed package is a blister pack in which the maximum moisture permeability is no more than 0.005 mg/day.

69. The package of claim 68, wherein the sealed package is a bottle and/or comprises an HDPE bottle.

70. The package of claim 69, wherein the bottle is closed with a heat induction liner.

71. The package of any one of claims 67-70, wherein the sealed package comprises an oxygen absorbing agent.

72. The package of claim 71, wherein the oxygen absorbing agent is iron.

73. The package of any one of claims 47-72, wherein the amount of laquinimod in the first composition is less than 0.6 mg.

74. The package of any one of claims 47-72, wherein the amount of laquinimod in the first composition is 0.1-40.0 mg.

75. The package of claim 74, wherein the amount of laquinimod is 0.1-2.5 mg.

76. The package of claim 74, wherein the amount of laquinimod is 0.25-2.0 mg.

77. The package of claim 74, wherein the amount of laquinimod is 0.5-1.2 mg.

78. The package of claim 74, wherein the amount of laquinimod is 0.25 mg, 0.3 mg, 0.5 mg, 0.6 mg, 1.0 mg, 1.2 mg, 1.5 mg or 2.0 mg.

79. The package of any one of claim 47-78, wherein the amount of the statin is 0.1-100 mg.

80. The package of claim 79, wherein the amount of the statin is 10-80 mg.

81. The package of claim 80, wherein the amount of the statin is about 10, 20, 40 or 80 mg.

82. The package of claim 81, wherein the amount of the statin is 10, 20, 40 or 80 mg.

83. The package of any one of claims 47-82, wherein the amount of laquinimod and the amount of the statin are prepared to be administered simultaneously, contemporaneously or concomitantly.

84. A therapeutic package for dispensing to, or for use in dispensing to, a subject afflicted with MS or presenting a CIS, which comprises:
a) one or more unit doses, each such unit dose comprising:
i) an amount of laquinimod and
ii) an amount of a statin
wherein the respective amounts of said laquinimod and said statin in said unit dose are effective, upon concomitant administration to said subject, to treat the subject, and
b) a finished pharmaceutical container therefor, said container containing said unit dose or unit doses, said container further containing or comprising labeling directing the use of said package in the treatment of said subject.

85. The therapeutic package of claim 84, wherein the respective amounts of said laquinimod and said statin in said unit dose when taken together is more effective to treat the subject than when compared to the administration of said laquinimod in the absence of the statin or the administration of the statin in the absence of said laquinimod.

86. The therapeutic package of claims of claims 84 or 85, wherein the statin is atorvastatin calcium.

87. A pharmaceutical composition comprising an amount of laquinimod and an amount of a statin.

88. The pharmaceutical composition of claim 86, consisting essentially of an amount of laquinimod and an amount of a statin.

89. The pharmaceutical composition of claims 87-88 for use in treating a subject afflicted with MS or presenting a CIS, wherein the laquinimod and the statin are prepared to be administered simultaneously, contemporaneously or concomitantly.

90. The pharmaceutical composition of any one of claims 87-89 wherein laquinimod is laquinimod sodium.

91. The pharmaceutical composition of any one of claims 87-90, wherein the statin is atorvastatin calcium.

92. The pharmaceutical composition of any one of claims 87-91, in an aerosol, an inhalable powder, an injectable, a liquid, a solid, a capsule or a tablet form.

93. The pharmaceutical composition of claim 92, wherein the tablets are coated with a coating which inhibits oxygen from contacting the core.

94. The pharmaceutical composition of claim 93, wherein the coating comprises a cellulosic polymer, a detackifier, a gloss enhancer, or pigment.

95. The pharmaceutical composition of any one of claims 87-94, further comprising mannitol, an alkalinizing agent, an oxidation reducing agent, a lubricant or a filler.

96. The pharmaceutical composition of claim 95, wherein the alkalinizing agent is meglumine.

97. The pharmaceutical composition of claims 95 or 96, wherein the lubricant is present in the composition as solid particles.

98. The pharmaceutical composition of any one of claims 95-97, wherein the lubricant is sodium stearyl fumarate or magnesium stearate.

99. The pharmaceutical composition of any one of claims 95-98, wherein the filler is present in the composition as solid particles.

100. The pharmaceutical composition of any one of claims 95-99, wherein the filler is lactose, lactose monohydrate, starch, isomalt, mannitol, sodium starch glycolate, sorbitol, lactose spray dried, lactose anhydrouse, or a combination thereof.

101. The pharmaceutical composition of claim 100, wherein the filler is mannitol or lactose monohydrate.

102. The pharmaceutical composition of any one of claims 87-101, which is free of an alkalinizing agent or an oxidation reducing agent.

103. The pharmaceutical composition of claim 102, which is free of an alkalinizing agent and free of an oxidation reducing agent.

104. The pharmaceutical composition of any one of claims 87-103, which is stable and free of disintegrant.

105. The pharmaceutical composition of any one of claims 87-104, wherein the amount of laquinimod in the composition is less than 0.6 mg.

106. The pharmaceutical composition of any one of claims 87-104, wherein the amount of laquinimod in the composition is 0.1-40.0 mg.

107. The pharmaceutical composition of claim 106, wherein the amount of laquinimod is 0.1-2.5 mg.

108. The pharmaceutical composition of claim 106, wherein the amount of laquinimod is 0.25-2.0 mg.

109. The pharmaceutical composition of claim 106, wherein the amount of laquinimod is 0.5-1.2 mg.

110. The pharmaceutical composition of claim 106, wherein the amount of laquinimod is 0.25 mg, 0.3 mg, 0.5 mg, 0.6 mg, 1.0 mg, 1.2 mg, 1.5 mg, 2.0 mg.

111. The pharmaceutical composition of any one of claim 87-110, wherein the amount of the statin is 0.1-100 mg.

112. The pharmaceutical composition of claim 110, wherein the amount of the statin is 10-80 mg.

113. The pharmaceutical composition of claim 110, wherein the amount of the statin is about 10, 20, 40 or 80 mg.

114. The pharmaceutical composition of claim 110, wherein the amount of the statin is 10, 20, 40 or 80 mg.

115. A pharmaceutical composition in unit dosage form, useful in treating a subject afflicted with MS or presenting a CIS, which comprises:
a) an amount of laquinimod;
b) an amount of a statin,
wherein the respective amounts of said laquinimod and said statin in said composition are effective, upon concomitant administration to said subject of one or more of said unit dosage forms of said composition, to treat the subject.

116. The pharmaceutical composition of claim 113, wherein the respective amounts of said laquinimod and the statin in said unit dose when taken together is more effective to treat the subject than when compared to the administration of said laquinimod in the absence of the statin or the administration of the statin in the absence of said laquinimod.

117. A pharmaceutical composition comprising an amount of laquinimod for use in treating a subject afflicted with MS or presenting a CIS as an add-on therapy or in combination with a statin.

118. A pharmaceutical composition comprising an amount of laquinimod for use in treating a subject afflicted with MS or presenting a CIS simultaneously, contemporaneously or concomitantly with a statin.

119. A pharmaceutical composition comprising an amount of a statin for use treating a subject afflicted with MS or presenting a CIS as an add-on therapy or in combination with laquinimod.

120. A pharmaceutical composition comprising an amount of a statin for use treating a subject afflicted with MS or presenting a CIS simultaneously, contemporaneously or concomitantly with laquinimod.

121. Laquinimod for use as an add-on therapy or in combination with a statin in treating a subject afflicted with MS or presenting a CIS.

122. A statin for use as an add-on therapy or in combination with laquinimod in treating a subject afflicted with MS or presenting a CIS.

123. Use of an amount of laquinimod and an amount of a statin in the preparation of a combination for treating a subject afflicted with MS or presenting a CIS wherein the laquinimod and the statin are prepared to be administered simultaneously, contemporaneously or concomitantly.

124. The pharmaceutical composition of any one of claims 115-120, or the use of any one of claims 121-123, wherein the statin is atorvastatin.

125. A process of preparing a pharmaceutical composition comprising an amount of laquinimod and an amount of a statin, comprising 1) obtaining an amount of laquinimod and an amount of a statin, and 2) admixing the laquinimod and the statin with a pharmaceutically acceptable carrier to make the pharmaceutical composition.

126. A process of preparing a pharmaceutical composition prepared for treating a subject afflicted with MS or presenting a CIS using an amount of laquinimod, either as an add-on therapy to or in combination with an amount of a statin, comprising 1) obtaining an amount of laquinimod, and 2) admixing the laquinimod with a pharmaceutically acceptable carrier.

127. Use of an amount of laquinimod in the manufacture of a medicament for treating a subject afflicted with MS or presenting a CIS wherein the laquinimod is prepared as an add-on therapy to or in combination with an amount of a statin, and wherein the amount of laquinimod and the amount of statin when taken together are effective to treat the subject.

128. Use of an amount of laquinimod and an amount of a statin in the manufacture of a medicament for treating a subject afflicted with MS or presenting a CIS, wherein the amount of laquinimod and an amount of statin when taken together are effective to treat the subject.

129. A process of preparing a medicament prepared for treating a subject afflicted with MS or presenting a CIS using an amount of laquinimod, either as an add-on therapy to or in combination with an amount of a statin, comprising 1) obtaining a pharmaceutical composition comprising an amount of laquinimod and a pharmaceutically acceptable carrier, and 2) packaging the pharmaceutical composition to make the medicament.

130. A process of preparing a medicament prepared for treating a subject afflicted with MS or presenting a CIS using an amount of laquinimod and an amount of a statin, comprising 1) obtaining a pharmaceutical composition comprising an amount of laquinimod, an amount of a statin, and a pharmaceutically acceptable carrier, and 2) packaging the pharmaceutical composition to make the medicament.
